(19) United States
(12) Patent Application Publication
(10) Pub. No.: US 2022/0135947 A1
(43) Pub. Date: May 5, 2022

(54) METHODS FOR CULTURING MESENCHYMAL STEM CELLS, PRODUCTS THEREOF, AND APPLICATIONS THEREOF

(71) Applicant: Pandorum Technologies Private Limited, Bangalore (IN)

(72) Inventors: Tuhin BHOWMICK, New York, NY (US); Arun CHANDRU, Bangalore (IN); Deepthi MENON, Bangalore (IN); Shivaram SELVAM, Hyderabad (IN); Midhun BEN THOMAS, Bangalore (IN); Wenson David RAJAN, Bangalore (IN)

(21) Appl. No.: 17/578,441

(22) Filed: Jan. 18, 2022

Related U.S. Application Data
(63) Continuation of application No. PCT/IN2020/050623, filed on Jul. 18, 2020.

(30) Foreign Application Priority Data
Jul. 18, 2019 (IN) 201941029039
Jul. 18, 2019 (IN) 201941029040
Jul. 18, 2019 (IN) 201941029041
Jul. 18, 2019 (IN) 201941029042

(51) Int. Cl. C12N 5/0775; C12N 5/073; C12N 5/00; A61K 35/28

(52) U.S. Cl. CPC C12N 5/0663; C12N 5/0605; C12N 5/0667; C12N 5/0062; C12N 2533/74; A61K 35/28; C12N 2500/98; C12N 2502/03; C12N 2513/00; C12N 5/0012

(57) ABSTRACT

The present disclosure provides a process for obtaining an expanded primed mesenchymal stem cell population. In the process, the MSCs are cultured in the culture medium comprising a corneal stromal stem cell derived-conditioned medium to obtain the expanded population of the primed mesenchymal stem cell population along with the mesenchymal stem cell derived-conditioned medium. Also, provided is a method of culturing the MSCs in 3D culture using a spheroid-based method or a microcarrier-based method, in order to obtain the expanded primed mesenchymal stem cell population. Further, an exosome preparation obtained from the expanded primed mesenchymal stem cell derived-conditioned medium is also disclosed herein. The present disclosure also discloses a composition comprising an expanded population of the primed mesenchymal stem cells, or a primed mesenchymal stem cell derived-conditioned medium, or an exosome preparation, or combinations thereof.

### FIELD OF INVENTION

The present disclosure broadly relates to the field of in-vitro cell culture, and particularly discloses methods for culturing mesenchymal stem cells for obtaining a population of expanded primed mesenchymal stem cells, and a mesenchymal stem cell derived-conditioned medium.

### BACKGROUND OF INVENTION

Multipotent mesenchymal stromal cells (MSC) are components of the tissue stroma of all adult organs that are located at perivascular sites. MSC plays a pivotal role in tissue homeostasis, surveillance, repair, and remodeling (Le Blanc K, Mougiakakos D. Multipotent mesenchymal stromal cells and the innate immune system. Nat Rev Immunol. 2012; 12:383-96). The therapeutic potential of MSCs isolated from different tissue sources is attributed to their ability to undergo lineage-specific differentiation, to modulate the immune system, and to secrete important bioactive factors. Due to the remarkable anti-inflammatory, immunosuppressive, immunomodulatory, and regenerative properties, the mesenchymal stem cells have garnered considerable attention in the field of the stem-cell based therapies. Various studies have already shown the promise that mesenchymal stem cell therapy hold in the management of various conditions like lung infections, neurological disorders, Parkinson's disease etc. MSCs also secrete exosomes that perform as mediators in the tumor niche and play several roles in tumorigenesis, angiogenesis, and metastasis. Exosomes also plays a very important role in intracellular communication.

The clinical applications of MSCs require reproducible cell culture methods and cell expansion methods that provide adequate numbers of cells of suitable quality and consistent therapeutic benefits. However, expansion of the MSCs to large quantities, is one of the perquisites of the cell-based therapies so as to empower the therapeutic efficacy of the MSC.

Accordingly, the current methods of culturing and expanding the yield of mesenchymal stem cells are not amenable to scale up the production of the MSCs or MSCs with high therapeutic efficacy.

Therefore, there is a dire need in the art to provide an improved and cost-effective method that not only allows the large-scale production of mesenchymal stem cells but also to amplify the yield of exosomes purified from the large-scale production of mesenchymal stem cells. The large number of mesenchymal stem cells and exosomes can then be further used in different cell-based therapies to address multiple unmet clinical needs.

### SUMMARY OF THE INVENTION

In an aspect of the present disclosure, there is provided a process for obtaining an expanded primed mesenchymal stem cell population, said process comprising: (a) obtaining a population of mesenchymal stem cells; (b) culturing the population of mesenchymal stem cells in a culture medium comprising a corneal stromal stem cell derived-conditioned medium, to obtain primed mesenchymal stem cells, wherein the corneal stromal stem cell derived-conditioned medium is obtained from culturing of corneal limbal stem cells; and (c) expanding the primed mesenchymal stem cells obtained in step (b) in a culture medium, to obtain an expanded primed mesenchymal stem cell population and a mesenchymal stem cell derived-conditioned medium.

In another aspect of the present disclosure, there is provided an expanded primed mesenchymal stem cell population obtained by the process as described herein.

In another aspect of the present disclosure, there is provided a mesenchymal stem cell derived-conditioned medium obtained by the process as described herein.

In another aspect of the present disclosure, there is provided a composition comprising the mesenchymal stem cell derived-conditioned medium as described herein.

In another aspect of the present disclosure, there is provided a composition comprising the expanded primed mesenchymal stem cell population as described herein.

In another aspect of the present disclosure, an exosome preparation obtained by a process comprising: (a) harvesting the mesenchymal stem cell derived-conditioned medium as described herein, to obtain a secretome; (b) centrifuging the secretome, to obtain a pellet; (c) dissolving the pellet in a low serum xenofree media, to obtain a crude solution; (d) performing density gradient ultracentrifugation with the crude solution, to obtain a fraction comprising exosomes; and (e) purifying the fraction comprising the exosomes by size exclusion chromatography, to obtain an exosome preparation.

In another aspect of the present disclosure, there is provided a composition comprising at least two components selected from the group consisting of: (a) the expanded primed mesenchymal stem cell population as described herein, (b) the mesenchymal stem cell derived-conditioned medium as described herein, and (e) the exosome preparation as described herein.

In another aspect of the present disclosure, there is provided a method for treating a condition selected from the group consisting of corneal disorders, liver fibrosis, and hyper-inflammatory conditions, said method comprising: (a) obtaining the exosomes as described herein; and (b) administering the exosomes to a subject for treating the condition.

In another aspect of the present disclosure, there is provided a method for treating a condition selected from the group consisting of corneal disorders, liver fibrosis, and hyper-inflammatory conditions, said method comprising: (a) obtaining the mesenchymal stem cell derived-conditioned medium as described herein; and (b) administering a therapeutically effective amount of the conditioned medium to a subject for treating the condition.

In another aspect of the present disclosure, there is provided a method for treating a condition selected from the group consisting of corneal disorders, liver fibrosis, and hyper-inflammatory conditions, said method comprising: (a) obtaining the expanded primed mesenchymal stem cell population as described herein; and (b) administering a therapeutically effective amount of the expanded primed mesenchymal stem cell population to a subject for treating the condition.

In another aspect of the present disclosure, there is provide a method for treating a condition selected from the group consisting of corneal disorders, liver fibrosis, and hyper-inflammatory conditions, said method comprising: (a) obtaining the composition as described herein; and (b) administering a therapeutically effective amount of the composition to a subject for treating the condition. These and other features, aspects, and advantages of the present subject matter will be better understood with reference to the following description and appended claims. This summary is provided to introduce a selection of concepts in a simplified form. This summary is not intended to identify key features or essential features of the claimed subject matter, nor is it intended to be used to limit the scope of the claimed subject matter.

### BRIEF DESCRIPTION OF ACCOMPANYING DRAWINGS

The following drawings form a part of the present specification and are included to further illustrate aspects of the present disclosure. The disclosure may be better understood by reference to the drawings in combination with the detailed description of the specific embodiments presented herein.

FIG. 1 depicts the four xeno-free methods applied for isolation and culturing of CSSCs, in accordance with an embodiment of the present disclosure.

FIG. 2 depicts the characterization of CSSCs isolated by the xenofree protocols as disclosed in the present disclosure; comparison of expression of CSSC specific markers (CD90/CD73/CD105) confirms the protocol employing Liberase for digestion and MEM media for culture as optimal for the xenofree culture of CSSCs; Scale bar: 100 μm, in accordance with an embodiment of the present disclosure.

FIG. 3 depicts the characterization of CSSCs isolated by LIB_MEM protocol in accordance with an embodiment of the present disclosure.

FIG. 4 depicts the characterization of hBM-MSCs (RoosterBio Inc.); Key: Lane 1: D200: Donor #200; Lane 2: D227: Donor 227; Lane 3: D257: Donor 257. Scale bar: 100 μm, in accordance with an embodiment of the present disclosure.

FIG. 5 depicts the characterization of immortalized adipose derived mesenchymal stem cells (ADMSC), in accordance with an embodiment of the present disclosure.

FIG. 6 depicts (A) CSSCs secrete more HGF than BMMSCs. CSSC priming (10% CSSC-CM & 25% CSSC-CM) modestly improved HGF secretion in BMMSC Donor #200. (B) BMMSCs secrete more IL-6 than CSSCs. CSSC priming (10% CSSC-CM & 25% CSSC-CM) decreased the IL-6 secretion by BMMSCs. Since it is only one donor, data is not conclusive. (C) CSSCs secrete less VEGF compared to all three BMMSC donors. (D) Nerve Growth factor (NGF) and soluble Fms Related Receptor Tyrosine Kinase 1 (sFLT1) were detected in CSSC secretome while BMMSC-secretome from three donors (ID #200, #227 and #257) did not express detectable levels of the proteins (by western blot). Priming of BMMSC Donor #200 with CSSC-CM induced the secretion of NGF and sFLT1 in the secretome at both 10% and 25% supplementation, in accordance with an embodiment of the present disclosure.

FIG. 7 depicts the schematic depiction of core crosslinked alginate beads (crosslinked with divalent or trivalent ions and their combinations thereof) possessing glutaraldehyde crosslinked gelatin to promote cell attachment, in accordance with an embodiment of the present disclosure.

FIG. 8 depicts the flowchart depicting the steps involved in the preparation of alginate microbeads crosslinked with Ca²⁺/Ba²⁺ ions with a cell adhesive gelatin crosslinked surface, in accordance with an embodiment of the present disclosure.

FIG. 9A depicts the phase contrast image of the microbeads, b) depicts the size distribution of the microbeads and c) depicts the circularity distribution profile. Scale bar 250 mm, in accordance with an embodiment of the present disclosure.

FIG. 10 depicts the Cell adherence and viability on fabricated Alg/Gel microbeads. a) Phase contrast image and b) Live dead assay on BM-MSC adhered microbeads 24 h after cell loading in static conditions. c) Phase contrast image of BM-MSCs and d) Live dead assay on BM-MSC adhered microbeads after static loading (24 h) and 72 h in dynamic condition. Scale bar: 200 mm, in accordance with an embodiment of the present disclosure.

FIG. 11 depicts the Live dead assay performed on a) PS beads, b) RCP beads and c) Alg/Gel microbeads. Dotted line represents outline of bead surface. Scale bar: 100 mm, in accordance with an embodiment of the present disclosure.

FIG. 12 depicts the Immunostaining for αSMA on a) PS beads, b) RCP beads and c) Alg/Gel microbeads. Lower αSMA expression (GREEN) was observed in Alg/Gel and RCP microcarriers compared to PS beads. (d-f) represents CD90 (RED) stem cell marker expression of cultured cells on PS, RCP and Alg/Gel microbeads. Dotted line represents outline of bead surface. Scale bar: 100 mm, in accordance with an embodiment of the present disclosure.

FIG. 13 depicts the microbeads of the present disclosure (Alg/Gel microbeads) with cells treated with dissolution buffer. a) at 0 mins, b) after 1 min, c) after 7 mins and d) cell viability assay using trypan blue demonstrating 80% viability. Scale bar: 200 mm, in accordance with an embodiment of the present disclosure.

FIG. 14 depicts the scheme depicting the generation of scalable MSC spheroids, in accordance with an embodiment of the present disclosure.

FIG. 15 depicts the A. Phase-contrast images taken 24 hr and 48 h after seeding the cells in the hanging drop with or without methylcellulose. B. Confocal images of viability staining from the spheroid from day 2 and 5 showing the minimal cell death in the spheroids cultured in both +methylcellulose and −methylcellulose. Scale bar: 200 μm, in accordance with an embodiment of the present disclosure.

FIG. 16 depicts the (A) Confocal images of viability staining from the spheroid at a seeding density of 1500 cells from day 4 showing minimal cell death in the spheroids cultured in both +methylcellulose and −methylcellulose (hanging drop method). Scale bar: 50 μm. (B) Confocal images of viability staining from the spheroid at an initial seeding density of 10,000 cells from day 4 showing minimal cell death in the spheroids cultured in both +methylcellulose and −methylcellulose (hanging drop method). Scale bar: 200 μm, in accordance with an embodiment of the present disclosure.

FIG. 17 depicts the A. Schematic summary of the experiment executed for the hanging drop-spinner flask culture of hBM-MSC spheroids. B. Phase-contrast microscopy images of spheroids taken on day 0 of static hanging drop culture, day 3 and day 7 in the spinner flask culture showing the compactness of the spheroids were well maintained during the culture period. C. Live-Dead staining performed on day 3 and day 7 in the spinner culture. D. Whole-spheroid immunofluorescence staining of CD90 (MSC marker) performed on day 7 of the spinner flask culture. E. Whole-spheroid immunofluorescence staining of alpha-SMA performed on day 7 of the spinner flask culture. Scale bar: 200 μm, in accordance with an embodiment of the present disclosure.

FIG. 18 depicts the Schematic summary of the experiment executed for the direct-spinner flask culture of hBM-MSC spheroids. B. Phase-contrast microscopy images of spheroids taken on day 2, 3 and 5 post-seeding in the spinner flask. C. Live-Dead staining on spheroids performed on day 2 and day. Scale bar: 200 μm, in accordance with an embodiment of the present disclosure.

FIG. 19 depicts the scheme for isolation of exosomes Iodixanol density gradient ultracentrifugation, in accordance with an embodiment of the present disclosure.

FIG. 20 depicts the secretory cytokine profile of BMMSCs and CSSCs in 2D culture. (A) BMMSCs secrete more IL-6 than CSSCs; (B) CSSCs secrete more HGF than BMMSCs. (C) CSSCs secrete less VEGF compared to all three BMMSC donors, in accordance with an embodiment of the present disclosure.

FIG. 21 depicts the comparison of exosome population isolated by Single step ultracentrifugation (UC_Step 1), 30% sucrose cushion and iodixanol gradient ultracentrifugation protocols: (A-C) Demonstrate the heterogeneity of the exosome particle size obtained in each method of purification. Single step UC purification of exosomes results in isolation of particles in the range of 50-170 nm, 30% sucrose cushion gives us particles in the range of 60-150 nm while iodixanol gives us a tighter range of 30-130 nm, in accordance with an embodiment of the present disclosure.

FIG. 22 depicts the Particle concentration of fraction 9 (F9): 1.8×10¹⁰/ml) (A and B); C. Median particle diameter in nm ranged between 100-150 nm; D. Avg. size distribution of F9: 28-133 nm. Particle size distribution and particle number were determined by NTA. Particles were detected at 11 different positions of the cell and averaged. Each sample was run in 3 technical replicates. E. Exosomes (fraction 9) isolated from hBM-MSCs were positive for typical exosome markers including CD63, CD9, CD81, ALIX and TSG101, in accordance with an embodiment of the present disclosure.

FIG. 23 depicts the Transmission Electron Microscopy (TEM) images of exosomes isolated by iodixanol density gradient ultracentrifugation. Lower magnification of representative images is shown in (A) and the respective magnified image (marked in yellow box) is shown in (B). Scale bars (0.2 um (E), and 200 nm (F)). The TEM images shows exosomes in the expected size range of about 150-250 nm range and complements the NTA data, in accordance with an embodiment of the present disclosure.

FIG. 24 depicts the Exosome size distribution and cargo characterization post size exclusion chromatography. (A-D) All fractions up to F7 were run on NTA. From F5, no particles were detected and only alternate fractions were run thereon. (E) Particle concentration per fraction (Fraction 9 was diluted into two fractions (2+3). (F) Flow cytometry analysis of fraction 2 and 3 from captocore purification identified 75% and 54% of the exosome population in fraction 2 and fraction 3 to be CD81/CD9 positive, respectively. (G) Western blot analysis of exosome markers CD81, CD9, CD63, ALIX and TSG101 in captocore purified fraction 9, in accordance with an embodiment of the present disclosure.

FIG. 25 depicts the Size distribution analysis of exosomes purified from BMMSCs by 30% cushion-based sucrose density method using Nano Tracking Analysis (NTA). A representative image of histogram is shown in A. The averaged data from 3 independent readings of size distribution are presented in B. (C) The total yield of exosomes from 30% sucrose cushion ultracentrifugation determined by NTA. (D). Western blot analysis for exosome marker CD9. Protein samples from secretome and exosome preparation were separated on a 12% SDS PAGE gel and antibody against CD9 was used to identify exosomes. CD9 was present both in secretome and exosome samples showing expected size of 24-27 Kda and the control samples were negative. (E and F) Transmission Electron Microscopy (TEM) images of exosomes isolated by 30% sucrose method. Lower magnification of representative images is shown in (E) and the respective magnified image (marked in yellow box) is shown in (F). Scale bars (0.2 um (E), and 200 nm (F)). The TEM images shows exosomes in the expected size range of about 150-250 nm range and complements the NTA data, in accordance with an embodiment of the present disclosure.

FIG. 26 depicts the Size distribution analysis of exosomes purified from CSSCs by 30% sucrose cushion density (30% SUC) based ultracentrifugation (A to C) and (D-E) iodixanol density gradient ultracentrifugation (IDX Fraction 9 (IDX-F9)) method using Nano Tracking Analysis (NTA). A representative image of histogram is shown in A, D for 30% SUC and IDX-F9 respectively. The averaged data from 3 independent readings of size distribution are presented in B &E for 30% SUC and IDX-F9 respectively. (C) The total yield of exosomes from 30% SUC and IDX-F9 respectively determined by NTA. (F) Western blot analysis for exosome marker CD9 for 30% SUC and IDX-F9. Protein samples from secretome and exosome preparation were separated on a 12% SDS PAGE gel and antibody against CD9 was used to identify exosomes. CD9 was present both in secretome and exosome samples showing expected size of 24-27 Kda and the control samples were negative, in accordance with an embodiment of the present disclosure.

FIG. 27 depicts the reproducibility of the exosome purification protocol (iodixanol density gradient ultracentrifugation) as disclosed in the present disclosure, in accordance with an embodiment of the present disclosure.

FIG. 28 depicts the comparison of purity of exosomes purified by three methods (i) single step ultracentrifugation (UC_step 1), (ii) s\30% sucrose cushion (iii) iodixanol gradient UC (IDX). (A) Sucrose cushion and iodixanol gradient methods gave comparable purity and low levels of VEGF compared to UC_Step 1 (single step ultracentrifugation) while retaining therapeutic factors such as HGF (B), in accordance with an embodiment of the present disclosure.

FIG. 29 depicts the comparison of scalability of CSSC-CM primed MSCs versus CSSC in clinical applications, in accordance with an embodiment of the present disclosure.

### DETAILED DESCRIPTION OF THE INVENTION

Those skilled in the art will be aware that the present disclosure is subject to variations and modifications other than those specifically described. It is to be understood that the present disclosure includes all such variations and modifications. The disclosure also includes all such steps, features, compositions, and compounds referred to or indicated in this specification, individually or collectively, and any and all combinations of any or more of such steps or features.

### Definitions

For convenience, before further description of the present disclosure, certain terms employed in the specification, and examples are delineated here. These definitions should be read in the light of the remainder of the disclosure and understood as by a person of skill in the art. The terms used herein have the meanings recognized and known to those of skill in the art, however, for convenience and completeness, particular terms and their meanings are set forth below.

The articles “a”, “an” and “the” are used to refer to one or to more than one (i.e., to at least one) of the grammatical object of the article.

The terms “comprise” and “comprising” are used in the inclusive, open sense, meaning that additional elements may be included. It is not intended to be construed as “consists of only”.

Throughout this specification, unless the context requires otherwise the word “comprise”, and variations such as “comprises” and “comprising”, will be understood to imply the inclusion of a stated element or step or group of element or steps but not the exclusion of any other element or step or group of element or steps.

The term “including” is used to mean “including but not limited to”. “Including” and “including but not limited to” are used interchangeably.

For the purposes of the present document, the term “a population of expanded primed mesenchymal stem cells” refers to the population of mesenchymal stem cells which has an increased number of cells as compared to the population of mesenchymal stem cells obtained initially for culturing. The culturing process does not differentiate the cells, it just increases the number of cells manifolds. The term “three-dimensional” or “3D” refers to a system of culturing the cells in-vitro in which the biological cells are allowed to grow and interact with their surroundings in all the three dimensions. The term “two-dimensional” or “2D” refers to the method of culturing the cells on a surface by which the biological cells are able to interact with their surroundings in two dimensions. The term “spheroid-based system” refers to the process of culturing mesenchymal stem cells (MSC) in a three-dimensional manner by formation of spheroids according to the method as described in the present disclosure. The term “microcarrier-based system” refers to the process of culturing mesenchymal stem cells (MSC) in a three-dimensional manner by the formation of alginate-gelatin (Alg/Gel) microcarriers or microbeads according to the method as described in the present disclosure. The term “microcarriers” and “microbeads” are used interchangeably, it refers to the alginate-gelatin (Alg/Gel) microcarriers or microbeads as described in the present disclosure. The term “mesenchymal stem cell derived-conditioned medium or “MSC-CM” refers to the medium obtained after the growth of the MSC. The conditioned medium thus obtained comprises secreted cell modulators and multiple factors critical for tissue regeneration. The conditioned medium thus obtained also comprises secretome, and exosomes which needs to be purified from the conditioned medium before being able to apply for therapeutic purposes. The process for obtaining expanded MSC as described herein also leads to the formation of MSC-CM, therefore, it can be said that a single process leads to the procurement of a population of expanded primed MSC as well as of MSC-CM. The term “exosomes” refers to the type of an extracellular vesicle that contain constituents (in terms of protein, DNA, and RNA) of the biological cells that secretes them. The exosomes obtained from the conditioned medium as described herein is used for therapeutic purposes.

For the purposes of the present document, the term “corneal limbal stem cells” refers to the population of stem cells which reside in the corneal limbal stem cell niche. The corneal limbal stem cell is referred to population of stem cells represented majorly by corneal stromal stem cells (CSSC), and limbal epithelial stem cells (LESC).

The term “corneal stromal stem cell derived-conditioned medium or “CSSC-CM” refers to the medium in which corneal stromal stem cells (CSSC) are grown. The CSSC-CM as described herein is obtained by culturing of CSSC in a manner known in the art or by culturing of CSSC as per the method disclosed herein.

The term “xeno-free” as described in the present disclosure refers to the process as described herein which is free of any product which is derived from non-human animal. The method being xeno-free is an important advantage because of its plausibility of clinical application. The term “scalable” refers to the ability to increase the production output manifolds. The term “subject” refers to a human subject who is suffering from the conditions as mentioned in the present disclosure. The term “therapeutically effective amount” refers to the amount of a composition which is required for treating the conditions of a subject.

The term “culture medium” refers to the medium in which the MSC is cultured. The culture medium comprises MSC basal medium, and the MSC basal medium is used as per the MSC which is being cultured. The MSC basal medium as mentioned in the present disclosure was commercially procured. For the purposes of the present disclosure, RoosterBio xenofree media was used for BMMSCs.

The term “low serum xeno free medium” refers to the standard xeno free medium which is low on the serum level which is commercially available for the purposes of culturing MSC. It can be contemplated that a person skilled in the art can use any such medium for the purposes of the present disclosure.

The term “primed mesenchymal stem cell” refers to the MSC which are primed with a corneal stromal stem cell derived-conditioned medium (CSSC-CM). The priming is done at several volume percentage of CSSC-CM with respect to the culture medium.

The term “expanded primed mesenchymal stem cell population” refers to the expanded population of the primed MSC. As per the present disclosure, the priming is done by CSSC-CM.

The term “culturing” broadly covers the expansion of cells also. The expansion allows the stem cells to multiply into same cell type without differentiating into subsequent cell lineages.

The term “population of mesenchymal stem cells” refers to the population of naive cells. The naïve cells here refer to the unprimed mesenchymal stems are not primed with any conditioned medium. Therefore, the terms unprimed and naïve are interchangeably used in the present disclosure.

In the present, the products derived from the cell culture methods as disclosed herein comprises the expanded (cultured) corneal stromal stem cell population which, conditioned medium derived from corneal stromal stem. The conditioned medium is further used to purify cell-derived products such as secretome, exosome, and other extracellular matrix (ECM) components like biopolymers. The cell-derived components are further used for the methods of treatment as disclosed herein and for various regenerative purposes. The process as described in the present disclosure is an in-vitro process, i.e. taking place in an artificially created environment outside of the living being.

Ratios, concentrations, amounts, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. For example, a volume percentage in a range of 5-50% range of about 5-50% should be interpreted to include not only the explicitly recited limits of about 5% to about 50%, but also to include sub-ranges, such as 5-45%, 15-50%, and so forth, as well as individual amounts, including fractional amounts, within the specified ranges, such as 5.5%, and 45.5%, for example.

The methods available in the literature for culturing and expansion of corneal stromal stem cells (CSSCs) have various limitations: (i) For CSSCs to be meet the increasing demands of clinical applications (for e.g., wound healing), fresh CSSCs are isolated. The step of isolation of fresh CSSCs from human donor makes the whole process very difficult for obtaining enriched population of CSSCs; (ii) The yield of CSSCs is very poor as compared to the MSCs derived from BMMSCs; (iv) The number of CSSCs obtained by the conventional methods are not sufficient to exhibit the enhanced therapeutic effect in terms of corneal wound healing; (v) The yield of secretory proteins, extracellular vesicle (EV), such as, exosomes derived from the enriched population of CSSCs is a limiting factor for large-scale production for stem cell therapies. Therefore, due to low yield of CSSCs, and exosomes derived from said CSSCs, their use is often limited in various clinical applications.

In order to address the problems faced in the art, the present disclosure provides a method for scalable production of enriched population of mesenchymal stem cells. The present disclosure provides a cost-effective and scalable method of priming mesenchymal stem cells with the CSSC-derived conditioned medium that skews the phenotype of BM-MSCs towards a more CSSC-like profile. The process of priming the MSCs with the CSSC-derived conditioned medium (CSSC-CM) helps to circumvent the need to isolate fresh CSSCs from human donor corneas, which are difficult to procure. Further, the process of the present disclosure helps to minimize donor to donor variation in exosome batch production. In an example of the present disclosure, the MSCs derived from human Bone marrow (BM-MSCs) are primed with the CSSC-CM. The process reprograms BM-MSCs to behave like CSSCs that helps in providing sufficient cell yield of CSSC-CM primed BM-MSCs, which can be then be efficiently used for various therapeutic applications. Moreover, the process of the present disclosure also helps in obtaining large amount of conditioned medium comprising enriched population of CSSC-CM primed BM-MSCs. Also, reprograming of BM-MSCs to behave like CSSCs provide sufficient cell yields for the production of therapeutic exosomes.

To evaluate the effect of the priming of BM-MSCs with the CSSC-CM on the yield of the final product (i.e., CSSC-CM primed BM-MSCs, or CSSC-CM primed BM-MSCs-derived conditioned medium, or exosomes-derived from CSSC-CM primed BM-MSCs or exosomes-derived from CSSC-CM primed BM-MSCs-derived conditioned medium) the yield of unprimed CSSCs (i.e., CSSCs not subjected to priming), and yield of unprimed CSSCs are evaluated and compared. In case of unprimed CSSCs, about 0.5-1 million stem cells per donor cornea can be expanded to 4-6 million cells up to 3 passages. On the contrary, the commercially available unprimed BMMSCs can be expanded from 1 million to 80-120 million in 3 passages (RoosterBio Inc.). Although, the yield of unprimed BMMSCs is 20-30 folds higher cell than the yield of unprimed CSSCs. However, the effect of CSSCs (cornea resident MSCs) for effectively healing the corneal wound, cannot be mimicked by the use of BMMSCs. Therefore, according to the present disclosure, the priming of BMMSCs with CSSC-conditioned media to reprogram BMMSCs into CSSC-like stem cells helps in producing 20-60 folds higher CSSC-like BMMSC cell yield and exosomes. While using CSSC-exosomes can only help treat 8-10 corneas at a dose of 0.1-0.5 billion exosomes per eye, the process of the present disclosure helps to treat 20-60× i.e. 200-600 patients from a single donor cornea. Furthermore, the three-dimensional (3D) scalable cell expansion process is also provided in the present disclosure, that helps to further amplify the cell and exosome yield by an additional 5-10 folds. As demonstrated in the present disclosure, the CSSC-CM primed BM-MSCs secretes high levels of HGF and low levels of VEGF and IL-6. Moreover, the process of the present disclosure when used in combination with the 3D expansion method helps to obtain 100-600 folds higher exosomes yield, thereby, allowing the treatment of approximately 1000-5000 patients per donor cornea. Overall, the present disclosure provides a viable, cost-effective, and less labor-intensive method to scale-up the production of MSC-derived exosomes that would help in meeting the current challenges faced in the art to obtain a high-quality yield of exosomes that can be used for various therapeutic applications.

Unless defined otherwise, all technical and scientific terms used herein have the same meaning as commonly understood by one of ordinary skill in the art to which this disclosure belongs. Although any methods and materials similar or equivalent to those described herein can be used in the practice or testing of the disclosure, the preferred methods, and materials are now described. All publications mentioned herein are incorporated herein by reference.

The present disclosure is not to be limited in scope by the specific embodiments described herein, which are intended for the purposes of exemplification only. Functionally-equivalent products, compositions, and methods are clearly within the scope of the disclosure, as described herein.

In an embodiment of the present disclosure, there is provided a process for obtaining an expanded primed mesenchymal stem cell population, said process comprising: (a) obtaining a population of mesenchymal stem cells; (b) culturing the population of mesenchymal stem cells in a culture medium comprising a corneal stromal stem cell derived-conditioned medium, to obtain primed mesenchymal stem cells, wherein the corneal stromal stem cell derived-conditioned medium is obtained from culturing of corneal limbal stem cells; and (c) expanding the primed mesenchymal stem cells obtained in step (b) in a culture medium, to obtain an expanded primed mesenchymal stem cell population and a mesenchymal stem cell derived-conditioned medium.

In an embodiment of the present disclosure, there is provided a process for obtaining a mesenchymal stem cell derived-conditioned medium, said process comprising: (a) obtaining a population of mesenchymal stem cells; (b) culturing the population of mesenchymal stem cells in a culture medium comprising a corneal stromal stem cell derived-conditioned medium, to obtain primed mesenchymal stem cells, wherein the corneal stromal stem cell derived-conditioned medium is obtained from culturing of corneal limbal stem cells; and (c) expanding the primed mesenchymal stem cells obtained in step (b) in a culture medium, to obtain an expanded primed mesenchymal stem cell population and a mesenchymal stem cell derived-conditioned medium.

In an embodiment of the present disclosure, there is provided a process for obtaining an expanded primed mesenchymal stem cell population, said process comprising: (a) obtaining a population of mesenchymal stem cells; (b) culturing the population of mesenchymal stem cells in a culture medium comprising a corneal stromal stem cell derived-conditioned medium in a volume percentage in a range of 5-50% with respect to the culture medium, to obtain primed mesenchymal stem cells, wherein the corneal stromal stem cell derived-conditioned medium is obtained from culturing of corneal limbal stem cells; and (c) expanding the primed mesenchymal stem cells obtained in step (b) in a culture medium, to obtain an expanded primed mesenchymal stem cell population and a mesenchymal stem cell derived-conditioned medium. In another embodiment of the present disclosure, the mesenchymal stem cells obtained in step (b) is contacted with a culture medium comprising a corneal stromal stem cell derived-conditioned medium in a volume percentage in a range of 10-40% with respect to the culture medium. In yet another embodiment of the present disclosure, the mesenchymal stem cells obtained in step (b) is contacted with a culture medium comprising a corneal stromal stem cell derived-conditioned medium in a volume percentage in a range of 15-30% with respect to the culture medium. In one another embodiment of the present disclosure, the mesenchymal stem cells obtained in step (b) is contacted with a culture medium comprising a corneal stromal stem cell derived-conditioned medium in a volume percentage in a range of 20-28% with respect to the culture medium.

In an embodiment of the present disclosure, there is provided a process for obtaining an expanded primed mesenchymal stem cell population, said process comprising: (a) obtaining a population of mesenchymal stem cells; (b) culturing the population of mesenchymal stem cells in a culture medium comprising a corneal stromal stem cell derived-conditioned medium, to obtain primed mesenchymal stem cells, wherein the corneal stromal stem cell derived-conditioned medium is obtained from culturing of corneal limbal stem cells; and (c) expanding the primed mesenchymal stem cells obtained in step (b) in a culture medium is done in either a spheroid-based system or a microcarrier-based system, to obtain an expanded primed mesenchymal stem cell population and a mesenchymal stem cell derived-conditioned medium. In another embodiment of the present disclosure, expanding the primed mesenchymal stem cells is done in a spheroid-based system. In yet another embodiment of the present disclosure, expanding the primed mesenchymal stem cells is done in a microcarrier-based system.

In an embodiment of the present disclosure, there is provided a process for obtaining an expanded primed mesenchymal stem cell population, said process comprising: (a) obtaining a population of mesenchymal stem cells; (b) culturing the population of mesenchymal stem cells in a culture medium comprising a corneal stromal stem cell derived-conditioned medium, to obtain primed mesenchymal stem cells, wherein the corneal stromal stem cell derived-conditioned medium is obtained from culturing of corneal stromal limbal cells; and (c) expanding the primed mesenchymal stem cells obtained in step (b) in a culture medium is done in either a spheroid-based system or a microcarrier-based system, to obtain an expanded primed mesenchymal stem cell population and a mesenchymal stem cell derived-conditioned medium, wherein expanding the mesenchymal stem cells is done in a spheroid-based system comprising steps of: (i) pelleting the primed mesenchymal stem cells obtained in step (b) as described herein, to obtain a primed mesenchymal stem cell pellet; (ii) resuspending the primed mesenchymal stem cell pellet in a suitable volume of a culture medium comprising MSC basal medium, to obtain a primed mesenchymal stem cell suspension; (iii) processing the primed mesenchymal stem cell suspension to obtain primed mesenchymal stem cell spheroids having a density of mesenchymal stem cells in a range of 600-10,000 cells per spheroid; and (iv) culturing the primed mesenchymal stem cell spheroids in a culture medium comprising MSC basal medium to obtain a population of expanded primed mesenchymal stem cells, and a mesenchymal stem cell derived-conditioned medium.

In an embodiment of the present disclosure, there is provided a process for obtaining an expanded primed mesenchymal stem cell population, said process comprising: (a) obtaining a population of mesenchymal stem cells; (b) culturing the population of mesenchymal stem cells in a culture medium comprising a corneal stromal stem cell derived-conditioned medium, to obtain primed mesenchymal stem cells, wherein the corneal stromal stem cell derived-conditioned medium is obtained from culturing of corneal limbal stem cells; and (c) expanding the primed mesenchymal stem cells obtained in step (b) in a culture medium is done in either a spheroid-based system or a microcarrier-based system, to obtain an expanded primed mesenchymal stem cell population and a mesenchymal stem cell derived-conditioned medium, wherein expanding the mesenchymal stem cells is done in a spheroid-based system comprising steps of: (i) pelleting the primed mesenchymal stem cells obtained in step (b) as described herein, to obtain a primed mesenchymal stem cell pellet; (ii) resuspending the primed mesenchymal stem cell pellet in a suitable volume of a culture medium comprising MSC basal medium, to obtain a primed mesenchymal stem cell suspension, wherein the culture medium comprises methyl cellulose in a concentration range of 0.2-2% with respect to the culture medium; (iii) processing the primed mesenchymal stem cell suspension to obtain primed mesenchymal stem cell spheroids having a density of mesenchymal stem cells in a range of 600-10,000 cells per spheroid; and (iv) culturing the primed mesenchymal stem cell spheroids in a culture medium comprising MSC basal medium to obtain a population of expanded primed mesenchymal stem cells, and a mesenchymal stem cell derived-conditioned medium, wherein the culture medium comprises methyl cellulose in a concentration range of 0.2-2% with respect to the culture medium. In another embodiment of the present disclosure, the culture medium of step (ii) and step (iv) comprises methyl cellulose in a concentration range of 0.5-1.8% with respect to the culture medium. In yet another embodiment of the present disclosure, the culture medium of step (ii) and step (iv) comprises methyl cellulose in a concentration range of 0.8-1.3% with respect to the culture medium.

In an embodiment of the present disclosure, there is provided a process for obtaining an expanded primed mesenchymal stem cell population, said process comprising: (a) obtaining a population of mesenchymal stem cells; (b) culturing the population of mesenchymal stem cells in a culture medium comprising a corneal stromal stem cell derived-conditioned medium, to obtain primed mesenchymal stem cells, wherein the corneal stromal stem cell derived-conditioned medium is obtained from culturing of corneal limbal stem cells; and (c) expanding the primed mesenchymal stem cells obtained in step (b) in a culture medium is done in either a spheroid-based system or a microcarrier-based system, to obtain an expanded primed mesenchymal stem cell population and a mesenchymal stem cell derived-conditioned medium, wherein expanding the mesenchymal stem cells is done in a spheroid-based system comprising steps of: (i) pelleting the primed mesenchymal stem cells obtained in step (b) as described herein, to obtain a primed mesenchymal stem cell pellet; (ii) resuspending the primed mesenchymal stem cell pellet in a suitable volume of a culture medium comprising MSC basal medium, to obtain a primed mesenchymal stem cell suspension, wherein the culture medium comprises methyl cellulose in a concentration range of 0.2-2% with respect to the culture medium; (iii) processing the primed mesenchymal stem cell suspension to obtain primed mesenchymal stem cell spheroids having a density of mesenchymal stem cells in a range of 600-10,000 cells per spheroid; and (iv) culturing the primed mesenchymal stem cell spheroids in a culture medium comprising MSC basal medium, to obtain a population of expanded primed mesenchymal stem cells, and a mesenchymal stem cell derived-conditioned medium.

In an embodiment of the present disclosure, there is provided a process for obtaining an expanded primed mesenchymal stem cell population, said process comprising: (a) obtaining a population of mesenchymal stem cells; (b) culturing the population of mesenchymal stem cells in a culture medium comprising a corneal stromal stem cell derived-conditioned medium, to obtain primed mesenchymal stem cells, wherein the corneal stromal stem cell derived-conditioned medium is obtained from culturing of corneal limbal stem cells; and (c) expanding the primed mesenchymal stem cells obtained in step (b) in a culture medium is done in either a spheroid-based system or a microcarrier-based system, to obtain an expanded primed mesenchymal stem cell population and a mesenchymal stem cell derived-conditioned medium, wherein expanding the mesenchymal stem cells is done in a spheroid-based system comprising steps of: (i) pelleting the primed mesenchymal stem cells obtained in step (b) as described herein, to obtain a primed mesenchymal stem cell pellet; (ii) resuspending the primed mesenchymal stem cell pellet in a suitable volume of a culture medium comprising MSC basal medium, to obtain a primed mesenchymal stem cell suspension; (iii) processing the primed mesenchymal stem cell suspension to obtain primed mesenchymal stem cell spheroids having a density of mesenchymal stem cells in a range of 600-10,000 cells per spheroid; (iv) culturing the primed mesenchymal stem cell spheroids in a culture medium comprising MSC basal medium to obtain a population of expanded primed mesenchymal stem cells, and a mesenchymal stem cell derived-conditioned medium, wherein the culture medium comprises methyl cellulose in a concentration range of 0.2-2% with respect to the culture medium.

In an embodiment of the present disclosure, there is provided a process for obtaining an expanded primed mesenchymal stem cell population, said process comprising: (a) obtaining a population of mesenchymal stem cells; (b) culturing the population of mesenchymal stem cells in a culture medium comprising a corneal stromal stem cell derived-conditioned medium, to obtain primed mesenchymal stem cells, wherein the corneal stromal stem cell derived-conditioned medium is obtained from culturing of corneal limbal stem cells; and (c) expanding the primed mesenchymal stem cells obtained in step (b) in a culture medium is done in either a spheroid-based system or a microcarrier-based system, to obtain an expanded primed mesenchymal stem cell population and a mesenchymal stem cell derived-conditioned medium, wherein expanding the primed mesenchymal stem is done in a microcarrier based system comprising steps of: (i) obtaining microcarriers comprising crosslinked alginate core and crosslinked gelatin surface; (ii) suspending the microcarriers in a culture medium, to obtain a suspension; (iii) seeding the suspension with the primed mesenchymal stem cells obtained in step (b) as described herein; and (iv) culturing the primed mesenchymal stem cells to obtain a population of expanded primed mesenchymal stem cells adhered to the microcarriers, and a mesenchymal stem cell derived-conditioned medium, and wherein population of expanded primed mesenchymal stem cells adhered to the microcarriers is contacted with a dissolution buffer comprising sodium chloride and trisodium citrate to obtain a population of expanded primed mesenchymal stem cells.

In an embodiment of the present disclosure, there is provided a process for obtaining an expanded primed mesenchymal stem cell population, said process comprising: (a) obtaining a population of mesenchymal stem cells; (b) culturing the population of mesenchymal stem cells in a culture medium comprising a corneal stromal stem cell derived-conditioned medium, to obtain primed mesenchymal stem cells, wherein the corneal stromal stem cell derived-conditioned medium is obtained from culturing of corneal limbal stem cells; and (c) expanding the primed mesenchymal stem cells obtained in step (b) in a culture medium is done in either a spheroid-based system or a microcarrier-based system, to obtain an expanded primed mesenchymal stem cell population and a mesenchymal stem cell derived-conditioned medium, wherein expanding the primed mesenchymal stem is done in a microcarrier based system comprising steps of: (i) obtaining microcarriers comprising crosslinked alginate core and crosslinked gelatin surface; (ii) suspending the microcarriers in a culture medium, to obtain a suspension; (iii) seeding the suspension with the primed mesenchymal stem cells obtained in step (b) as described herein; and (iv) culturing the primed mesenchymal stem cells to obtain a population of expanded primed mesenchymal stem cells adhered to the microcarriers, and a mesenchymal stem cell derived-conditioned medium, and wherein the microcarriers are in a size ranging from 50-500 μm. In another embodiment of present disclosure, the microcarriers are in a size ranging from 100-450 μm. In yet another embodiment of the present disclosure, the microcarriers are in a size ranging from 150-350 μm. In one another embodiment of the present disclosure, the microcarriers are in a size ranging from 200-300 μm.

In an embodiment of the present disclosure, there is provided a process for obtaining an expanded primed mesenchymal stem cell population, said process comprising: (a) obtaining a population of mesenchymal stem cells; (b) culturing the population of mesenchymal stem cells in a culture medium comprising a corneal stromal stem cell derived-conditioned medium, to obtain primed mesenchymal stem cells, wherein the corneal stromal stem cell derived-conditioned medium is obtained from culturing of corneal limbal stem cells; and (c) expanding the primed mesenchymal stem cells obtained in step (b) in a culture medium is done in either a spheroid-based system or a microcarrier-based system, to obtain an expanded primed mesenchymal stem cell population and a mesenchymal stem cell derived-conditioned medium, wherein expanding the primed mesenchymal stem is done in a microcarrier based system comprising steps of: (i) obtaining microcarriers comprising crosslinked alginate core and crosslinked gelatin surface; (ii) suspending the microcarriers in a culture medium, to obtain a suspension; (iii) seeding the suspension with the primed mesenchymal stem cells obtained in step (b) as described herein; and (iv) culturing the primed mesenchymal stem cells to obtain a population of expanded primed mesenchymal stem cells adhered to the microcarriers, and a mesenchymal stem cell derived-conditioned medium, and wherein the microcarriers comprise sodium alginate in the concentration range of 0.01-20% w/v, and gelatin in the concentration range of 0.1-20% w/v. In another embodiment of the present disclosure, the microcarriers comprise sodium alginate in the concentration range of 0.1-19% w/v, and gelatin in the concentration range of 0.5-19% w/v. In yet embodiment of the present disclosure, the microcarriers comprise sodium alginate in the concentration range of 2-15% w/v, and gelatin in the concentration range of 5-15% w/v.

In an embodiment of the present disclosure, there is provided a process for obtaining an expanded primed mesenchymal stem cell population, said process comprising: (a) obtaining a population of mesenchymal stem cells; (b) culturing the population of mesenchymal stem cells in a culture medium comprising a corneal stromal stem cell derived-conditioned medium, to obtain primed mesenchymal stem cells, wherein the corneal stromal stem cell derived-conditioned medium is obtained from culturing of corneal limbal stem cells; and (c) expanding the primed mesenchymal stem cells obtained in step (b) in a culture medium is done in either a spheroid-based system or a microcarrier-based system, to obtain an expanded primed mesenchymal stem cell population and a mesenchymal stem cell derived-conditioned medium, wherein expanding the primed mesenchymal stem is done in a microcarrier based system comprising steps of: (i) obtaining microcarriers comprising crosslinked alginate core and crosslinked gelatin surface; (ii) suspending the microcarriers in a culture medium, to obtain a suspension; (iii) seeding the suspension with the primed mesenchymal stem cells obtained in step (b) as described herein; and (iv) culturing the primed mesenchymal stem cells to obtain a population of expanded primed mesenchymal stem cells adhered to the microcarriers, and a mesenchymal stem cell derived-conditioned medium, and wherein the microcarriers are in a size ranging from 50-500 μm, and wherein the microcarriers comprise sodium alginate in the concentration range of 0.01-20% w/v, and gelatin in the concentration range of 0.1-20% w/v.

In an embodiment of the present disclosure, there is provided a process for obtaining an expanded primed mesenchymal stem cell population, said process comprising: (a) obtaining a population of mesenchymal stem cells; (b) culturing the population of mesenchymal stem cells in a culture medium comprising a corneal stromal stem cell derived-conditioned medium, to obtain primed mesenchymal stem cells, wherein the corneal stromal stem cell derived-conditioned medium is obtained from culturing of corneal limbal stem cells; and (c) expanding the primed mesenchymal stem cells obtained in step (b) in a culture medium, to obtain an expanded primed mesenchymal stem cell population and a mesenchymal stem cell derived-conditioned medium, wherein culturing the population of mesenchymal stem cells in a culture medium is done in either a spheroid-based system or a microcarrier-based system.

In an embodiment of the present disclosure, there is provided a process for obtaining an expanded primed mesenchymal stem cell population, said process comprising: (a) obtaining a population of mesenchymal stem cells; (b) culturing the population of mesenchymal stem cells in a culture medium comprising a corneal stromal stem cell derived-conditioned medium, to obtain primed mesenchymal stem cells, wherein the corneal stromal stem cell derived-conditioned medium is obtained from culturing of corneal limbal stem cells; and (c) expanding the primed mesenchymal stem cells obtained in step (b) in a culture medium, to obtain an expanded primed mesenchymal stem cell population and a mesenchymal stem cell derived-conditioned medium, wherein culturing the population of mesenchymal stem cells in a culture medium is done in either a spheroid-based system or a microcarrier-based system.

In an embodiment of the present disclosure, there is provided a process for obtaining an expanded primed mesenchymal stem cell population, said process comprising: (a) obtaining a population of mesenchymal stem cells; (b) culturing the population of mesenchymal stem cells in a culture medium comprising a corneal stromal stem cell derived-conditioned medium, to obtain primed mesenchymal stem cells, wherein the corneal stromal stem cell derived-conditioned medium is obtained from culturing of corneal limbal stem cells; and (c) expanding the primed mesenchymal stem cells obtained in step (b) in a culture medium, to obtain an expanded primed mesenchymal stem cell population and a mesenchymal stem cell derived-conditioned medium, wherein culturing the population of mesenchymal stem cells in a culture medium is done in spheroid-based system comprising the steps of: (i) pelleting the primed mesenchymal stem cells obtained in step (b) as described herein, to obtain a primed mesenchymal stem cell pellet; (ii) resuspending the primed mesenchymal stem cell pellet in a suitable volume of a culture medium comprising MSC basal medium, to obtain a primed mesenchymal stem cell suspension; (iii) processing the primed mesenchymal stem cell suspension to obtain primed mesenchymal stem cell spheroids having a density of mesenchymal stem cells in a range of 600-10,000 cells per spheroid; (iv) culturing the primed mesenchymal stem cell spheroids in a culture medium comprising MSC basal medium to obtain a population of expanded primed mesenchymal stem cells, and a mesenchymal stem cell derived-conditioned medium, and wherein the culture medium of step (ii) and step (iv) comprises methyl cellulose in a concentration range of 0.2-2% with respect to the culture medium.

In an embodiment of the present disclosure, there is provided a process for obtaining an expanded primed mesenchymal stem cell population, said process comprising: (a) obtaining a population of mesenchymal stem cells; (b) culturing the population of mesenchymal stem cells in a culture medium comprising a corneal stromal stem cell derived-conditioned medium, to obtain primed mesenchymal stem cells, wherein the corneal stromal stem cell derived-conditioned medium is obtained from culturing of corneal limbal stem cells; and (c) expanding the primed mesenchymal stem cells obtained in step (b) in a culture medium, to obtain an expanded primed mesenchymal stem cell population and a mesenchymal stem cell derived-conditioned medium, wherein culturing the population of mesenchymal stem cells in a culture medium is done in a microcarrier based system comprising steps of: (i) obtaining microcarriers comprising crosslinked alginate core and crosslinked gelatin surface; (ii) suspending the microcarriers in a culture medium, to obtain a suspension; (iii) seeding the suspension with the primed mesenchymal stem cells obtained in step (b) as described herein; and (iv) culturing the primed mesenchymal stem cells to obtain a population of expanded primed mesenchymal stem cells adhered to the microcarriers, and a mesenchymal stem cell derived-conditioned medium, wherein the microcarriers are in a size ranging from 50-500 μm, and wherein the microcarriers comprise sodium alginate in the concentration range of 0.01-20% w/v, and gelatin in the concentration range of 0.1-20% w/v.

In an embodiment of the present disclosure, there is provided a process for obtaining an expanded primed mesenchymal stem cell population, said process comprising: (a) obtaining a population of mesenchymal stem cells; (b) culturing the population of mesenchymal stem cells in a culture medium comprising a corneal stromal stem cell derived-conditioned medium, to obtain primed mesenchymal stem cells, wherein the corneal stromal stem cell derived-conditioned medium is obtained from culturing of corneal limbal stem cells; and (c) expanding the primed mesenchymal stem cells obtained in step (b) in a culture medium, to obtain an expanded primed mesenchymal stem cell population and a mesenchymal stem cell derived-conditioned medium, wherein the corneal stromal stem cell derived-conditioned medium is obtained by culturing of corneal limbal stem cells, said culturing comprises: (i) obtaining a limbal ring tissue from a human donor cornea; (ii) mincing the tissue, to obtain fragments in the size ranging from 1 to 2 mm; (iii) suspending the fragments in an incomplete medium, to obtain a suspension; (iv) subjecting the fragments to digestion in the presence of at least one type of collagenase enzyme at a concentration range of 5-20 IU/μl with respect to the suspension, to obtain digested explants; (v) culturing the digested explants in a complete medium comprising 1-3% human platelet lysate for a period of 10-14 days, to obtain a population of corneal limbal stem cells; and (vi) passaging the corneal limbal stem cells of step (v) for a period of 10-14 days, to obtain expanded corneal stromal stem cells and a corneal stromal stem cell derived-conditioned medium. In another embodiment of the present disclosure, mincing the tissue, to obtain fragments in the size ranging from 1.2 to 1.8 mm, or 1.4 to 1.6 mm, and wherein the at least one type of collagenase enzyme has a concentration range of 8-18 IU/μl with respect to the suspension

In an embodiment of the present disclosure, there is provided a process for obtaining an expanded primed mesenchymal stem cell population, said process comprising: (a) obtaining a population of mesenchymal stem cells; (b) culturing the population of mesenchymal stem cells in a culture medium comprising a corneal stromal stem cell derived-conditioned medium to obtain primed mesenchymal stem cells, wherein the corneal stromal stem cell derived-conditioned medium is obtained from culturing of corneal limbal stem cells; and (d) expanding the primed mesenchymal stem cells obtained in step (c) in a culture medium, to obtain an expanded primed mesenchymal stem cell population and a mesenchymal stem cell derived-conditioned medium, wherein the population of mesenchymal stem cells is selected from the group consisting of human bone marrow-derived mesenchymal stem cells, adipose tissue-derived mesenchymal stem cells, umbilical cord-derived mesenchymal stem cells, Wharton jelly-derived mesenchymal stem cells, dental pulp derived mesenchymal stem cells, and induced pluripotent stem cells. In another embodiment of the present disclosure, the population of mesenchymal stem cells is human bone marrow-derived mesenchymal stem cells.

In an embodiment of the present disclosure, there is provided an expanded primed mesenchymal stem cell population obtained by the process as described herein.

In an embodiment of the present disclosure, there is provided a mesenchymal stem cell derived-conditioned medium obtained by the process as described herein.

In an embodiment of the present disclosure, there is provided a composition comprising the mesenchymal stem cell derived-conditioned medium as described herein.

In an embodiment of the present disclosure, there is provided a composition comprising the expanded primed mesenchymal stem cell population as described herein.

In an embodiment of the present disclosure, there is provided an exosome preparation obtained by a process comprising: (a) harvesting the mesenchymal stem cell derived-conditioned medium as described herein, to obtain a secretome; (b) centrifuging the secretome, to obtain a pellet; (c) dissolving the pellet in a low serum xenofree media, to obtain a crude solution; (d) performing density gradient ultracentrifugation with the crude solution, to obtain a fraction comprising exosomes; and (e) purifying the fraction comprising the exosomes by size exclusion chromatography, to obtain an exosome preparation.

In an embodiment of the present disclosure, there is provided a composition comprising at least two components selected from the group consisting of: (a) the expanded primed mesenchymal stem cell population as described herein, (b) the mesenchymal stem cell derived-conditioned medium as described herein, and (e) the exosome preparation as described herein.

In an embodiment of the present disclosure, there is provided a method for treating a condition selected from the group consisting of corneal disorders, liver fibrosis, and hyper-inflammatory conditions, said method comprising: (a) obtaining the exosomes as described herein; and (b) administering the exosomes to a subject for treating the condition.

In an embodiment of the present disclosure, there is provided a method for treating a condition selected from the group consisting of corneal disorders, liver fibrosis, and hyper-inflammatory conditions, said method comprising: (a) obtaining the mesenchymal stem cell derived-conditioned medium as described herein; and (b) administering a therapeutically effective amount of the conditioned medium to a subject for treating the condition.

In an embodiment of the present disclosure, there is provided a method for treating a condition selected from the group consisting of corneal disorders, liver fibrosis, and hyper-inflammatory conditions, said method comprising: (a) obtaining the expanded primed mesenchymal stem cell population as described herein; and (b) administering a therapeutically effective amount of the expanded primed mesenchymal stem cell population to a subject for treating the condition.

In an embodiment of the present disclosure, there is provided a method for treating a condition selected from the group consisting of corneal disorders, liver fibrosis, and hyper-inflammatory conditions, said method comprising: (a) obtaining the composition as claimed in claim 19; and (b) administering a therapeutically effective amount of the composition to a subject for treating the condition. In an embodiment of the present disclosure, there is provided a composition comprising the mesenchymal stem cell derived-conditioned medium as described herein, for use in treating a condition selected from the group consisting of corneal disorders, liver fibrosis, and hyper-inflammatory conditions.

In an embodiment of the present disclosure, there is provided a composition comprising the expanded primed mesenchymal stem cell population for use in treating a condition selected from the group consisting of corneal disorders, liver fibrosis, and hyper-inflammatory conditions.

In an embodiment of the present disclosure, there is provided a composition comprising at least two components selected from the group consisting of: (a) the expanded primed mesenchymal stem cell population as described herein, (b) the mesenchymal stem cell derived-conditioned medium as described herein, and (e) the exosome preparation as described herein, for use in treating a condition selected from the group consisting of corneal disorders, liver fibrosis, and hyper-inflammatory conditions.

In an embodiment of the present disclosure, there is provided the expanded mesenchymal stem cell population as described herein, for use in treating a condition selected from the group consisting of corneal disorders, liver fibrosis, and hyper-inflammatory conditions.

In an embodiment of the present disclosure, there is provided the mesenchymal stem cell derived-conditioned medium as described herein, for use in treating a condition selected from the group consisting of corneal disorders, liver fibrosis, and hyper-inflammatory conditions.

In an embodiment of the present disclosure, there is provided the exosome preparation as described herein, for use in treating a condition selected from the group consisting of corneal disorders, liver fibrosis, and hyper-inflammatory conditions.

In an embodiment of the present disclosure, there is provided a process for obtaining an expanded primed mesenchymal stem cell population, said process comprising: (a) obtaining a population of mesenchymal stem cells; (b) culturing the population of mesenchymal stem cells in a culture medium comprising a corneal stromal stem cell derived-conditioned medium, to obtain primed mesenchymal stem cells, wherein the corneal stromal stem cell derived-conditioned medium is obtained from culturing of corneal limbal stem cells; and (c) expanding the primed mesenchymal stem cells obtained in step (b) in a culture medium, to obtain an expanded primed mesenchymal stem cell population and a mesenchymal stem cell derived-conditioned medium. In one implementation of the present disclosure, the population of mesenchymal stem cells are cultured by number of passages or subcultures. It can be contemplated that the population of mesenchymal stem cells are always cultured in the culture medium comprising corneal stromal stem cell derived-conditioned medium having a concentration in the range of 5-50% with respect to the culture medium, until the cells reach confluency. In another implementation of the present disclosure, the population of mesenchymal stem cells are always cultured in the culture medium comprising corneal stromal stem cell derived-conditioned medium having a concentration in the range of 5-50% for a time period in the range of 24-96 hours prior to confluency, wherein the xeno-free basal mesenchymal stem cell media is replaced with corneal stromal stem cell derived-conditioned medium.

Although the subject matter has been described in considerable detail with reference to certain examples and implementations thereof, other implementations are possible.

### EXAMPLES

The disclosure will now be illustrated with working examples, which is intended to illustrate the working of disclosure and not intended to take restrictively to imply any limitations on the scope of the present disclosure. Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices and materials are described herein. It is to be understood that this disclosure is not limited to particular methods, and experimental conditions described, as such methods and conditions may apply.

### Materials and Methods

### Source of Stem Cells

For the purpose of the present disclosure, mesenchymal stem cells derived from the sources such as bone marrow (BM), corneal limbal stem cells, umbilical cord (UC), Wharton's jelly (WJ), dental pulp (DP) and adipose tissue (AD), corneal limbal stem cell-derived conditioned media primed MSCs can be used in the methods and cell-derived products as described herein. The choice of the stem cell type would be target indication and tissue specific.

Source of Immortalized Adult Stem Cell Lines (Non-Viral Immortalized MSC Cell Lines):

1. Telomerized human Bone marrow derived mesenchymal stem cell line (BM-MSC/TERT277) was developed from mesenchymal stem cells isolated from spongy bone (sternum) by non-viral gene transfer of a plasmid carrying the hTERT gene. Positively transfected cells were selected by using neomycin phosphotransferase as selectable marker and Geneticin sulfate addition. The cell line was continuously cultured for more than 25 population doublings without showing signs of growth retardation or replicative senescence.
2. Telomerized human Wharton's Jelly derived mesenchymal stem cell line (WJ-MSC/TERT273) was established under xeno-free conditions from primary tissue disaggregation to non-viral transfer of hTERT.

The cell lines were characterized by unlimited growth while maintaining expression of cell type specific markers and functions such as: (i) typical mesenchymal morphology; (ii) expression of typical mesenchymal stem cell markers such as CD73, CD90 and CD105; (iii) differentiation potential towards adipocytes, chondrocytes, osteoblasts; and (iv) production of extracellular vesicles with angiogenic and anti-inflammatory activity.

Culture medium used—The culture medium used for culturing the mesenchymal stem cells comprises low serum xenofree medium supplemented with human platelet lysate (0-2%) and combination of 1-2 mM Glutamine, human Epidermal Growth Factor (1-50 ng/ml), Insulin, Transferrin, Selenium, Platelet derived growth Factor (10-100 ng/ml), bFibroblast Growth Factor (1-50 ng/ml), Hydrocortisone (10-100 mM), dexamethasone (0.01-1 mM), Ascorbic acid-2-phosphate (0.01-1 mM), and Insulin Growth Factor (1-50 ng/ml).

Minimum Essential medium—The MEM used for the culturing of CSSC comprises MEM along with low serum xenofree medium supplemented with human platelet lysate (0-2%) and combination of 1-2 mM Glutamine, human Epidermal Growth Factor (1-50 ng/ml), Insulin, Transferrin, Selenium, Platelet derived growth Factor (10-100 ng/ml), bFibroblast Growth Factor (1-50 ng/ml), Hydrocortisone (10-100 mM), dexamethasone (0.01-1 mM), Ascorbic acid-2-phosphate (0.01-1 mM), and Insulin Growth Factor (1-50 ng/ml).

### Media Composition

| Cell type | Components |
|---|---|
| BMMSC, ADMSC, | Combination of one or more of: Commercially available |
| DPMSC, UCSMC, WJMSC | media described below + (1-10%) and combination of 1-2 mM |
| | Glutamine, Insulin, Transferrin, Selenium, Platelet derived |
| | growth Factor (10-100 ng/ml), bFibroblast Growth Factor (1- |
| | 50 ng/ml) |
| CSSC, LESC | Combination of one or more of: Commercially available media |
| | described below + (1-10%) and combination of 1-2 mM |
| | Glutamine, human Epidermal Growth Factor (1-50 ng/ml), |
| | Insulin, Transferrin, Selenium, Platelet derived growth Factor |
| | (10-100 ng/ml), bFibroblast Growth Factor (1-50 ng/ml), |
| | Hydrocortisone (10-100 mM), dexamethasone (0.01-1 mM), |
| | Ascorbic acid-2-phosphate (0.01-1 mM), Insulin Growth Factor |
| | (1-50 ng/ml) |
| Commercially available | MEM (Gibco), DMEM (high or low glucose) (Gibco), Eagle's |
| media for all cell types | basal medium, Ham's F10 medium (F10) (Gibco), Ham's F-12 |
| including iPSCs | medium (F12) (Gibco), Iscove's modified Dulbecco's medium |
| | (IMDM) (Gibco), Liebovitz's L-15 medium, MCDB, |
| | DMEM/F12(Gibco), RPMI 1640 (Gibco), advanced DMEM |
| | (Gibco), DMEM/MCDB201 (Sigma), and CELL-GRO FREE, |
| | Mesenchymal Stem Cell Growth Medium (MSCGM), |
| | Mesencult-ACF Plus (StemCell Technologies), EpiLife ™ CF |
| | Kit (Gibco) |

### Example 1

### Culture and Expansion of Human Corneal Stromal Stem Cells (CSSC)

The present example describes the process for isolating, and culturing the corneal limbal stem cells, and enriching the stem cells to obtain a population of expanded corneal stromal stem cells (CSSC) under the xenofree culture conditions. CSSCs are type of MSCs derived from the tissues of cornea. The two major sub-populations of corneal limbal stem cells are CSSC and limbal epithelial stem cells (LESC). The process as disclosed in the present disclosure specifically enriches the heterogenous population of CSSC and LESC obtained in passage 1 to obtain an enriched and expanded population of CSSC.

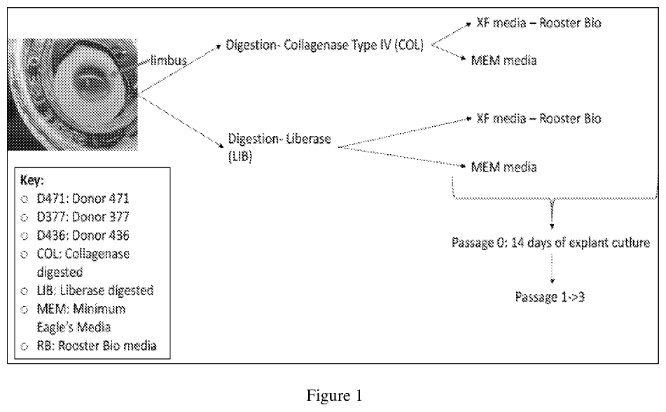

FIG. 1 shows the xenofree process for isolation and culturing CSSC from the human donor derived single cornea. The xenofree process for isolation and culture of CSSCs from human donor derived single cornea was optimized by testing four variations of xenofree culture protocols, where four different combinations of enzymes for digestion and media for culture were deployed (FIG. 1). The main aim was to select the combination of enzyme for digestion and media for culture that would result in obtaining the high-quality yield of CSSCs and high yield of exosomes. For this purpose, following combinations of collagenase enzyme and incomplete media were tested to evaluate the effectiveness of each combination for the isolation of CSSCs from human donor cornea:

(A): Combination I (LIB_MEM): Digestion with Liberase (LIB)+Minimum Essential Medium (MEM) media (Centre of Cellular Therapy (cGMP) validated).
(B): Combination II (LIB RB): Digestion with Liberase (LIB)+RoosterBio Xenofree Basal media (RB)
(C) Combination III (COL_RB): Digestion with Collagenase Type IV (COL)+RoosterBio Xenofree Basal media (RB)
(D) Combination IV (COL MEM): Digestion with Collagenase Type IV (COL)+MEM media (Centre of Cellular Therapy (cGMP) validated) (MEM).

The enzyme Liberase as described herein, is a type of collagenase enzyme, which is a combination of collagenase-I and collagenase-II.

To obtain the expanded corneal stromal stem cell population, the present disclosure describes a process for isolating and culturing corneal stem cells using a combination of liberase (collagenase enzymatic digestion) and MEM enzyme under xenofree conditions. The steps of the process are provided below:

(a) Human donor derived corneas were washed with antibiotic fortified buffered saline before extracting limbus which contain the CSSC.
(b) Under aseptic conditions, a 3600 limbal ring tissue was excised from the human donor cornea using surgical instruments.
(c) The excised limbal ring tissue was then washed with buffered saline and minced into smaller fragments.
(d) The minced tissue fragments were suspended into incomplete media (MEM or DMEM media) to obtain a suspension.
(e) The minced tissue fragments were subjected to collagenase digestion by adding 20 μL of reconstituted collagenase IV (17104019, Thermofisher) or Liberase (Roche) at a concentration of 5-20 IU/μL with respect to the tissue suspension, to obtain digested explants.
(f) After 16 h of incubation, collagenase enzymatic digestion was stopped by adding 2 mL of complete media fortified with 2% human platelet lysate (HPL).
(g) The digested explants were then spun down at 1000 rpm for 3 min at room temperature, in saline added with penicillin and streptomycin.
(h) At passage 0, the digested explants were resuspended in 5 mL xenofree complete media (MEM+2% HPL, 1× Insulin-Transferrin-Selenium (ITS), 10 ng/ml Epidermal growth factor (EGF)) and were cultured in Corning CellBIND flasks for 14 days to obtain the population of high quality corneal stromal stem cells for 14 days. The complete media was changed every 3 days.
(i) At the end of 14 days of passage 1 (P1), the cells isolated from the digested explants were trypsinized with Tryple (1×, Gibco) and resuspended in fresh complete media. The cells were seeded at 10,000 cells/cm² in CellBIND flasks for passages 1 through passage 2 (P2). The cells were then sub-cultured every 5-7 days each.

The expanded high quality CSSCs obtained at P1 and P2 were then characterized using the following markers: (i) Limbal epithelial stem cells (LESC) positive markers: p63a, ABCB5; (ii) Corneal stromal stem cells (CSSC) positive markers: CD90, CD73, CD105, ABCG2; and (iii) CSSC negative markers: a-SMA, CD34, ABCB5, p63-alpha.

For further characterization of CSSC, p63-alpha and ABCB5 (which are Limbal epithelial stem cell (LESC) population markers) were used for demonstrating the purity of the CSSC population isolated by the LIB_MEM process and the enrichment of CSSCs over LESCs from Passage 1 to Passage 2.

### Results

As described above, four combinations of collagenase enzyme and media were tested to evaluate the effectiveness of each combination to obtain high-quality yield of CSSCs from human donor cornea. The CSSCs obtained from each process deploying different combinations of collagenase enzyme and media were characterized based on the expression of CSSC-specific markers (CD90/CD73/CD105).

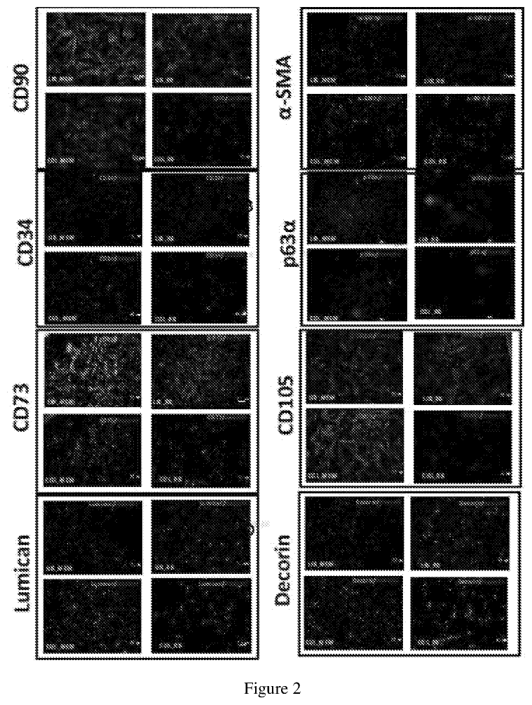

FIG. 2 shows the comparison between the four xenofree process using different combinations to obtain a high-quality yield of CSSCs, wherein the comparison was made in term of the expression of CSSC-specific markers in the CSSC population from each process. Referring to FIG. 2, the CSSCs consistently stained strongly positive for markers including CD90, CD73, CD105 and negative for alpha-SMA, CD34, decorin and lumican for CSSCs isolated by the process using the combination of LIB_MEM (combination 1). The other three processes (i.e., with combination II, III, IV) showed inconsistency in expression across cells and showed relatively lower expression of the positive markers (CD90, CD73, CD105). Therefore, it can be inferred that the process using the combination of LIB_MEM (combination 1) was found to be most suitable for the maintenance of stemness markers in CSSCs, as compared to processes using the combination II, III, and IV respectively.

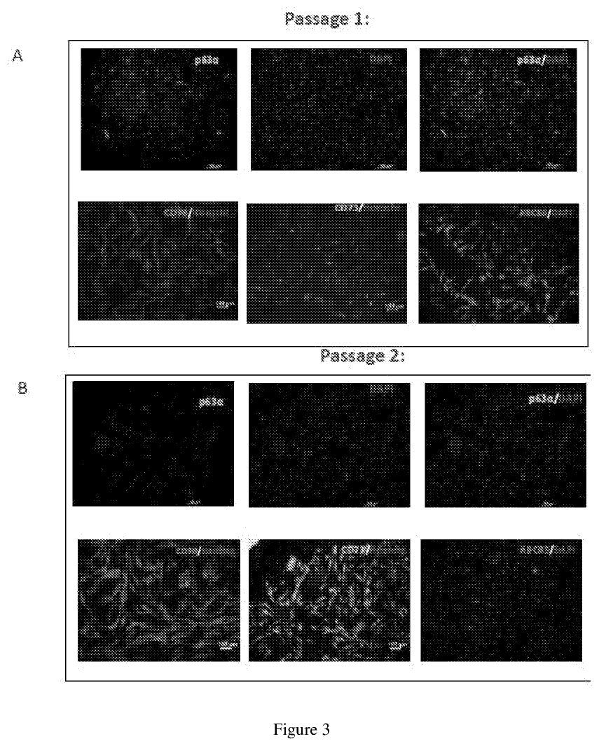

The CSSCs isolated and cultured by the process using the combination of LIB_MEM (combination I) were further characterized, as shown in FIG. 3. The process using the combination II yielded a mix of p63a/ABCB5 positive and negative cells at Passage 1 (FIG. 3A), indicating a mixed population of LESCs (positive stained) and CSSCs (negative stained). CD90 and CD73 were expressed by the stem cells in both passages. The number of CSSC obtained at passage 1 was in the range of 0.5-1 million.

However, at the passage 2 (FIG. 3B), the loss of expression of p63a-alpha and ABCB5, and high expression of CD90 and CD73 in CSSCs indicated the enrichment of CSSCs over LESCs. The enrichment of CSSCs over LESCs resulted in a pure stromal stem cell population. The yield of pure stromal stem cell population obtained at passage 3 was in the range of 4-6 million. The liberase enzyme as used herein is a combination of collagenase-I and collagenase-II in a ratio range of 0.3:1 to 0.5:1 along with a neutral protease content in a range of 1.8-2.6 mg. The collagenase-I content is in a range of 2.2-3.4 mg and the collagenase-II content is in a range of 1.5-2.3 mg which can be used.

Therefore, it can be inferred from FIG. 2 and FIG. 3 that the isolation and culture of CSSC using the combination I (LIB_MEM) resulted in high-quality yield of CSSCs. The high-quality yield of CSSC can then be further used for the production of high yield of secretomes and exosomes. The high population of CSSC and CSSC-derived secretomes and exosomes can be then used individually and in combination thereof, as a final product for various clinical applications from Passage 2-3.

### Example 2

Culture and Expansion of Primary Human Bone Marrow-Mesenchymal Stromal Cells (hBM-MSC)

The present example describes the process for culturing and expansion of hBM-MSC (RoosterBio Inc.) obtained from three donors (Donor ID #D200, D227 and D257). The expanded population of hBM-MSCs were further used for secretome and exosome production. The steps of the process for culturing and expansion of hBM-MSC was carried out by the following:
(a) The hBM-MSC High Performance Media Kit XF was kept at room temperature.
(b) The booster vial and media bottle well were sprayed with 70% isopropyl alcohol before transferring them into biosafety cabinet. The wet surface was wiped with a clean tissue paper.
(c) 1 vial (10 ml) hMSC Media Booster XFM (SU-016) was added to 500 ml hMSC High Performance Basal Media (SU-005) by using a serological pipette. Both the media was mixed with the pipettor. About 5-8 ml of complete media was added in to booster vial and was then gently mixed to retain any residual components of the booster.
(d) RoosterVial-hBM-1M-XF was obtained from liquid nitrogen (LN) and was immediately thawed in 37° C. water bath with gentle swirling. The process was monitored. RoosterVial-hBM-1M-XF was then removed from water bath after 2-3 min once the ice was melted.
(e) The vial was sprayed well with 70% isopropyl alcohol before transferring it into the biosafety cabinet. The cells were then aseptically transferred into a 50 mL centrifuge tube.
(f) 4 mL of culture media was slowly added dropwise to the cells in the centrifuge tube.
(g) The centrifuge tube was then centrifuged at 200×g for 10 min at room temperature.
(h) The supernatant was carefully removed without disturbing the cell pellet. The cells were then resuspended in 5 mL of complete media.
(i) As a quality control (QC), the cell number was counted and recorded.
(j) After resuspended the cells, the volume was made up to 30 mL with culture media.
(k) The media was mixed properly with the cells, and subsequently the cells were equally seeded into flasks, and more media was added to bring the volume up to the final volume to ensure that the fully coverage of the flask with the media.
(l) The flask was then transferred into a 5% CO₂, 37° C. sterilized incubator.
(m) The culture was microscopically observed every day from day 3 onwards to determine percentage confluency. If culture was found to be less than 50% confluent on day 3, then it led to the change in the media. The spent media was completely removed from the vessel and was replaced with the same volume of the fresh complete media. The vessel was transferred back into the incubator. When culture was found to be >80% confluent, harvesting of the cells was done on the following day.
(n) The media was changed on day 3 followed by every 48 h.
(o) For harvesting, the vessel was transferred into the biosafety cabinet and the spent media was removed. About 10 mL of spent media was collected in sterile container if it was used to quench harvest enzyme.
(p) The media was then removed, and the cells were washed with 1×PBS followed by addition of 10 mL of TrypLE and incubation in 37° C. incubator. The culture was checked every 5 min until the detachment of cells from the surface.
(q) Equal amount of quench (fresh media) or spent media was added to stop the TrypLE activity.
(r) The suspension was then transferred into a sterile 50 ml centrifuge tube. Subsequently, the centrifuge tube was centrifuged at 200×g for 10 min.
(s) The supernatant was aspirated, and the cells were resuspended with 4-5 mL of fresh media. The total volume of cell suspension was then measured.
(t) The well was mixed properly, and 0.1 mL of cells were transferred into microcentrifuge tubes for cell counts. The cells were diluted to 0.5 mL with DPBS to achieve the count of the cells in the range of 0.1-1×10⁶ cells/mL. The well was mixed, and cells were ready for counting with cell counting device.

Using this procedure, the cells can be expanded to 200 million (first passage) and up to 2 billion (second passage). The expanded hBM-MSC were further characterized using the stem cell markers CD90, CD73, CD105, alpha-SMA, and CD34.

### Result

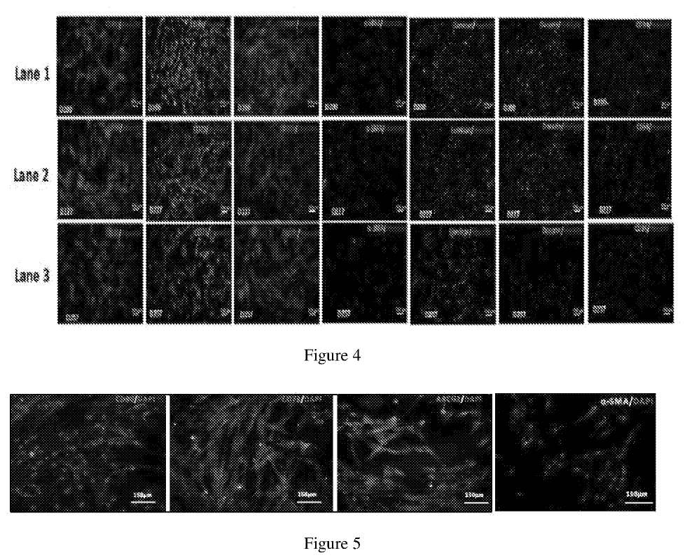

Human BM-MSCs (RoosterBio Inc.) from three donors (Donor ID #D200, D227 and D257) were cultured and expanded for secretome and exosome production, according to the process described above. The human BM-MSCs were characterized prior to exosome induction to confirm the stemness and integrity of the cells (quality check step). FIG. 4 shows the characterization of human BM-MSCs. Referring to FIG. 4, it can be observed that all three Human BM-MSCs stained positive for MSC markers including CD90, CD73, CD105 and negative for alpha-SMA, CD34. The human BM-MSCs expressed low levels of lumican and decorin (extracellular matrix proteins).

Therefore, it can be inferred from FIG. 4 that a high-quality yield of human BM-MSCs with positive expression of CD90, CD73, CD105 and negative expression of alpha-SMA, CD34. The expanded Human BM-MSCs was further used for the production of high yield of secretomes and exosomes. These human BM-MSC and human BM-MSCs-derived secretomes and exosomes can be then used individually and in combination thereof, as a final product for various clinical applications.

### Example 3

### Culture and Expansion of Adipose-Derived Mesenchymal Stem Cells (ADMSC)

The immortalized/telomerised ADMSCs (Cat #ASC/TERT1) were procured from Evercyte and cultured and expanded according to the process described in Example 2, however, Evercyte proprietary xenofree media was used instead of Rooster Bio media. The expanded ADMSCs were characterized using the cell markers CD90, CD73 and ABCG2, and alpha-SMA.

### Results

FIG. 5 shows the characterization of immortalized ADMSCs. Referring to FIG. 5, sternness markers, such as, CD90, CD73 and ABCG2 were expressed by the ADMSCs while stress marker alpha-SMA was not expressed by ADMSCs. The positive expression of markers such as CD90, CD73 and ABCG2 and negative expression of alpha-SMA indicates the isolation and expansion of high-quality yield of ADMSCs population. The expanded ADMSCs were further used for the production of high yield of secretomes and exosomes. These ADMSCs and ADMSC-derived secretomes and exosomes can be then used individually and in combination thereof, as a final product for various clinical applications.

### Example 4

### Culture & Expansion of Umbilical Cord Derived Mesenchymal Stromal Cells (UCMSC)

The present example describes the process for culturing and expansion of umbilical cord-derived mesenchymal stromal cells.

In this process, fresh Umbilical cords (UCs) were obtained from informed, healthy mothers in local maternity hospitals after normal deliveries and processed immediately. The cords were then rinsed twice in phosphate buffered saline in penicillin and streptomycin, and the cord blood was removed during the process. The washed cords were cut into 1-mm2 pieces and floated in low-glucose Dulbecco's modified Eagle's medium containing 10% fetal bovine serum. The pieces of cord were incubated at 37° C. in a humidified atmosphere consisting of 5% CO₂. Nonadherent cells were removed by washing. The medium was replaced every 3 days after the initial plating. When well-developed colonies of fibroblast-like cells appeared after 10 days, the cultures were trypsinized and passaged into a new flask for further expansion. UCMSCs from passage 2-5 were used for clinical applications.

### Example 5

### Culture & Expansion of CSSC-Conditioned Media (CSSC-Cm) Primed Mesenchymal Stem Cells and its Application

The present example explains the process of priming of the mesenchymal stem cells with the conditioned media derived from CSSC (CSSC-CM). CSSCs (cornea resident MSCs) is highly effective in corneal wound healing. This priming process helps in reprogramming of the mesenchymal stem cells to behave like CSSCs. The priming of mesenchymal stem cells with the CSSC-conditioned media helps to circumvent the need to isolate fresh CSSCs from human donor corneas for the production of CSSCs and CSSCs-derived exosomes, which are difficult to procure. Moreover, the primed mesenchymal stem cells also help in minimizing donor to donor variation in exosome batch production. Additionally, the yield of CSSCs is also very poor, when compared to commercially available sources of MSCs. Therefore, the process of priming of the MSCs with the conditioned media derived from CSSCs results in the production of a higher population of CSSCs-liked MSCs (primed MSCs). The high population of primed BM-MSCs can be further used for the production of high-quality yield of exosomes that can be further used for various therapeutic applications.

The MSCs derived from the sources such as, bone marrow, umbilical cord, adipose tissue, dental pulp, wharton's jelly) can be primed with the conditioned media derived from CSSCs. One of the implementations of the present disclosure describing the process of priming the MSCs derived from bone marrow (BMMSCs) with the conditioned media derived from CSSCs is explained in the present disclosure. It can be contemplated that the same process is applied for priming the MSCs derived from other sources also, and in obtaining the conditioned media-derived from MSCs.

(i) Process of Priming of the BMMSCs with the Conditioned Media Derived from CSSC

The process of priming of the BMMSCs with the conditioned media derived from CSSC was done by the following method:

(a) The CSSC-conditioned media (CSSC-CM) was obtained by the culturing the CSSCs isolated from a single cornea, by following the steps as described in the Example 1.
(b) The BMMSCs were cultured and expanded according to the process as described in Example 2.
(c) The BMMSCs obtained in step (b) were cultured in the presence of CSSC-CM in a concentration range of 5-50%. In particular, the BMMSCs were cultured in the presence of CSSC-CM at a concentration of 10% and 20%. It is noteworthy to mention here that BMMSCs were cultured from the passage 1 till the BMMSCs reached confluency, i.e., BMMSCs were always cultured in the presence of CSSC-CM. In another implementation of the present disclosure, BMMSCs were cultured in the presence of CSSC-CM in the concentration range of 5-50% for a time period in a range of 24-96 hours prior to confluency, i.e., the xenofree basal MSC media was replaced with CSSC-CM supplemented media for 24-96 hr prior to when the BMMSCs reached more than 90% confluency.
(d) The expansion of the primed BM-MSCs obtained in step (c) was done as per the culture protocol described in Example 2. The expansion of the primed BMMSCs can also be done by the protocol well known to a person skilled in the art. As per one of the implementations of the present disclosure, the expansion of the primed BM-MSCs can also be done as per the three-dimensional (3D) based methods as disclosed in the Examples 6 (alginate-gelatin microcarriers), and Example 7 (spheroid-based).

The expanded cells were incubated in serum-free media for 24 hours and conditioned media-derived from primed BMMSCs were then cultured for further processing.

### (ii) Comparison of the Scalability of CSSC-CM Primed BMMSCs Vs Unprimed CSSCs, and Unprimed BMMSCs

To demonstrate the benefits of the priming of the MSCS (BMMSCs) with the CSSCs conditioned media, characterization of CSSC-CM primed BMMSCs was done. For this purpose, the levels of Vascular endothelial growth factor (VEGF), Hepatocyte growth factor (HGF) and IL-6 secreted by unprimed CSSC, unprimed BMMSC, and primed CSSC-CM primed BMMSCs, were quantified and compared with each other.

For this purpose, the unprimed CSSCs and unprimed BMMSCs were cultured according to the process described in Example 1 and 2, respectively. The CSSC-CM primed BMMSCs were cultured according to the process as described in (i) above. Cells were incubated in serum-free media for 24 hours and conditioned media was collected for processing from unprimed CSSCs, unprimed BMMSCs, and CSSC-CM primed BMMSCs. Secretome of BMMSCs from three independent donors (#200, #227, #257) were harvested alongside CSSCs and CSSC-primed BMMSC (only Donor #200) and secreted levels of VEGF, HGF and IL-6 were quantified and compared. Since the CSSC-conditioned media contains HGF, therefore, controls were run wherein BMMSC-CM was spiked with 10% and 25% CSSC-CM prior to assaying.

### Results

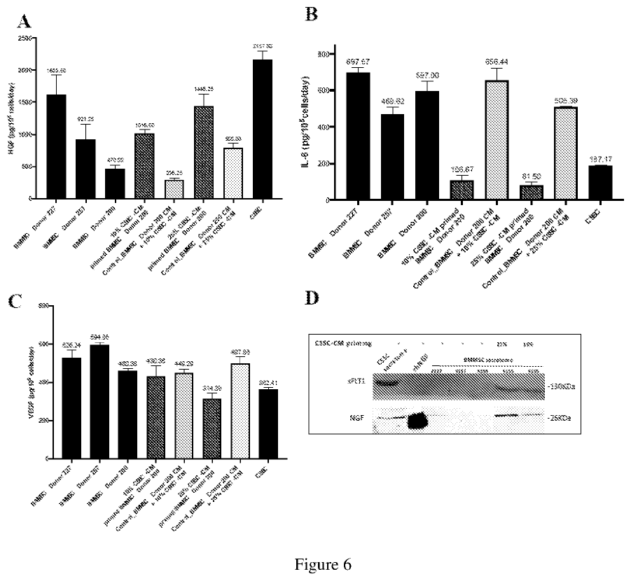

FIG. 6 shows the effect of priming BMMSC with the CSSC-conditioned media. Referring to FIG. 6A, CSSCs expressed more HGF levels than BMMSC. On the contrary, the levels of HGF secreted by CSSC-CM primed BMMSCs (with 10% CSSC-CM & 25% CSSC-CM) were modestly increased when compared to unprimed BMMSCs (from donor #200). Further, referring to FIG. 6B, CSSCs were found to secrete significantly lower levels of pro-inflammatory IL-6 compared to BMMSCs while priming of BMMSCs with CSSC-CM resulted in a marked decrease in the level of IL-6 secreted by the primed BMMSCs. From FIG. 6C, it can be observed that BMMSCs from all three donors secreted more VEGF than CSSCs alone. On the contrary, the levels of VEGF were reduced in CSSC-CM primed BMMSCs (Donor #200) in a dose dependent manner.

Since CSSC-conditioned media contains HGF, the control were run wherein BMMSC-CM was spiked with 10% and 25% CSSC-CM prior to assaying. As shown by the clear grey bars in the FIG. 6, the additive HGF values were quantified in the controls. Therefore, as shown in FIG. 6D, the controls demonstrated that the priming effects on HGF were not due to the additive or dilution effects of CSSC-CM+BMMSC-CM.

Moreover, it can also be observed that Nerve Growth factor (NGF) and soluble Fms Related Receptor Tyrosine Kinase 1 (sFLT1) were detected in CSSC secretome while BMMSC-secretome from three donors (ID #200, #227 and #257) did not express detectable levels of the proteins (by western blot). However, it was observed that the priming of BMMSC Donor #200 with CSSC-CM induced the secretion of NGF and sFLT1 in the secretome at both 10% and 25% supplementation.

A dose dependent response by the CSSC-CM primed BM-MSC can be observed as per the FIG. 6, therefore, the priming of BM-MSC is favourable in obtaining primed BM-MSC which are re-programmed to behave more like CSSC.

Therefore, it can be inferred from the above observations that priming BMMSCs with CSSC-CM skews the phenotype of BMMSC to behave more like CSSCs. The effect of priming with the CSSC-CM also applies to the MSCs derived from non-ocular sources such as AD-MSCs (Adipose-derived Mesenchymal stem cells). On integrating into the corneal microniche, the AD-MSCs modify their phenotype and secretory profile to behave more like corneal stromal stem cells. Therefore, this study explains the possibility of priming the MSCs derived from several sources (BM-UC-, AD-, DP, WJ-) with CSSC-CM for reprogramming these MSCs to behave more like CSSCs, so that these CSSC-CM primed MSCs can be further used for various clinical applications along with the exosomes derived from CSSC-CM primed MSCs. Consequently, this helps to reduce the dependence on a continuous supply of fresh donor corneas for the production of CSSCs and derived exosomes for clinical applications.

(iii) Use of the CSSC-CM Primed BMMSC

The process of priming of the BMMSCs with the CSSC-conditioned media not only helps in reprogramming of the BMMSCs into CSSC-like stem cells, but also helps in circumventing the need to isolate fresh CSSCs from human donor corneas, which are difficult to procure and also minimizes donor to donor variation in exosome batch production. Although the FIG. 6 depicts the data of expansion of CSSC-CM primed BM-MSC by the 2D method as described in the Example 5 and the advantage conferred by the priming. It can be contemplated that the advantage will be manifolds if the expansion is done by the 3D culture methods as disclosed in the Examples 6 and 7 of the present disclosure.

Also, the step of culturing the cells during priming of BM-MSC by the CSSC-CM (i.e. before the expansion of primed BM-MSCs) can be done by applying the 3D cell culture methods as disclosed in the Examples 6 and 7 of the present disclosure. Any person skilled in the art can use a combination of the 2D and 3D cell culture methods as disclosed herein to arrive at the successful expansion of primed BM-MSC and consequently harvest the secretome and exosome for clinical applications.

In the case of the unprimed CSSC, about 0.5-1 million stem cells per donor cornea were expanded to 4-6 million at the final passage 3. On the contrary, commercially available BMMSCs (RoosterBio Inc.) were expanded from 1 million to 80-120 million stem cell at the at the final passage 3. Hence, the yield of BMMSC was 20-30 folds higher than the yield of CSSCs. Further, when CSSC-derived exosomes were used for corneal applications, CSSC-derived exosomes were only able to treat 8-10 corneas at a dose of 0.1-0.5 billion exosomes per eye.

Even though the yield of BMMSC was higher than the yield of CSSC, the BMMSCs cannot mimic the use of CSSC for effective wound healing. Therefore, for this purpose, the priming of BMMSCs with CSSC-conditioned media was done to reprogram BMMSCs into CSSC-like stem cells. The process of the priming of the BMMSC with the CSSC-derived conditioned medium helps in the production of 20-60 folds higher CSSC-like BMMSC cell yield and exosomes. While CSSC-derived exosomes were only able to treat 8-10 corneas at a dose of 0.1-0.5 billion exosomes per eye, however, the priming process of the present disclosure helps to treat 20-60× i.e. 200-600 patients from a single donor cornea.

Therefore, it can be inferred from the above observations that the process of priming of the BMMSCs with the conditioned media derived from CSSC helps in the production of high-quality yield of CSSC-CM primed BMMSC and also helps in the production of condition medium-derived from CSSC-CM primed BMMSC. Moreover, the process also helps in the high-quality yield of exosomes as one of the final products of the present disclosure. The high-quality yield of CSSC-CM primed BMMSC, condition medium-derived from CSSC-CM primed BMMSC, and CSSC-CM primed BMMSC-derived exosomes can be used individually and in combinations thereof for various clinical applications.

### Example 6

### Expanding Stem Cells in Three-Dimensional (3D) Microcarrier-Based Culture

### Fabrication of Alginate-Gelatin Microcarriers

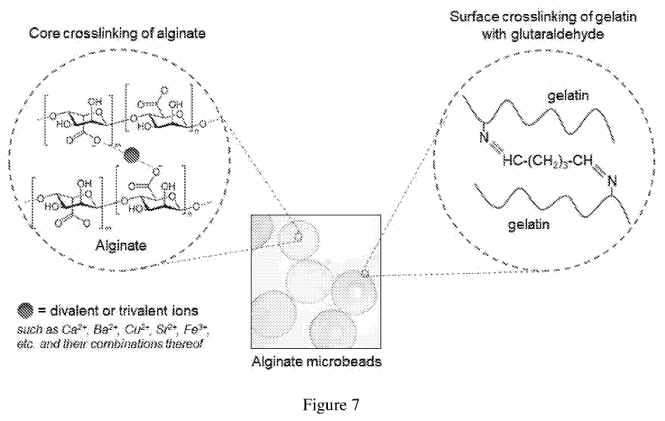

FIG. 7 depicts the basic concept behind the preparation of Alg/Gel microbeads for 3D culture of cells. Briefly, sodium alginate beads are fabricated by using commonly employed di- or trivalent ions as crosslinking agents, such as Ca²⁺ Ba²⁺, Fe²⁺, Cu²⁺, Sr²⁺, Fe³⁺, or their combinations thereof, to yield solid transparent microspheres. Subsequently, the microbeads ware coated with gelatin which will be reversibly crosslinked with glutaraldehyde. The gelatin coated bead surface facilitates cell adhesion and proliferation as bare alginate beads do no possess cell binding motifs conducive for cell adhesion and growth. Table 1 depicts the different components along with their percentages for obtaining the microcarriers/microbeads.

TABLE 1

| S.No. | Components/Parameters | Working ranges | |
|---|---|---|---|
| 1. | Sodium alginate (low/medium/high | 0.01-20% | w/v |
| | viscosity) | | |
| 2. | Di- or trivalent ions (Ca²⁺, | 0.01-1000 | mM |
| | Ba²⁺, Fe²⁺, Cu²⁺, Sr²⁺, Fe³⁺, | | |
| | and their combinations thereof) | | |
| 3. | EDTA | 0.1-100 | mM |
| 4. | Gelatin (50-400) bloom | 0.1-20% | w/v |
| 5. | Glutaraldehyde | 0.01-10% | v/v |
| 6. | Glycine | 1-1000 | mg/mL |
| 7. | Crosslinking time | 10 s-60 min | |
| 8. | Bead size (diameter) | 50-500 | μm |

As per one of the embodiments, the microcarriers that were synthesised for the present disclosure is as per the below mentioned protocol. Microcarriers—Alginate beads crosslinked with Ca2+ and Ba2+ ions and gelatin crosslinked with glutaraldehyde

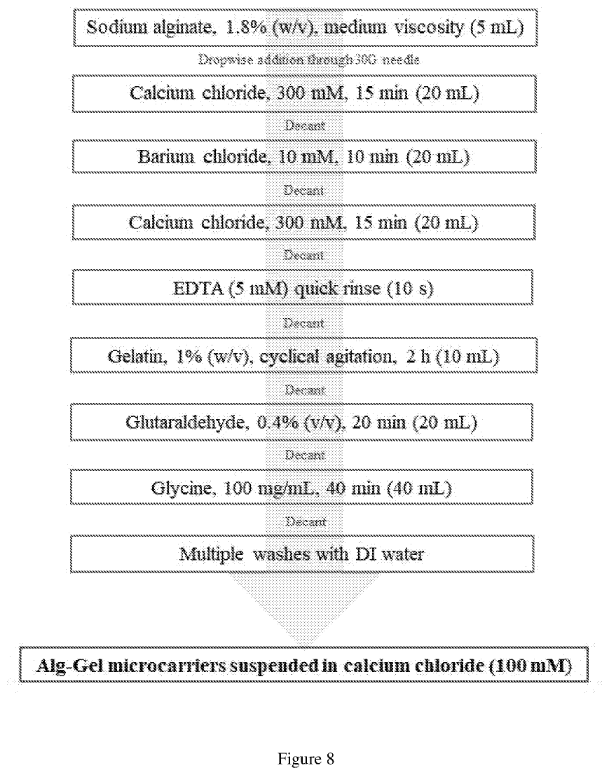

FIG. 8 depicts a flowchart for obtaining the alginate-gelatin based microcarriers used in the present disclosure. As one of the example, the alginate-gelatin based microcarrier system was developed using medium viscosity alginate. Briefly, alginate solution (1.8% w/v) was extruded from a 30 G needle into a bath containing calcium chloride solution (300 mM) to crosslink alginate. The crosslinking occurs due to the ionic interaction between the carboxyl groups of two adjacent alginate chains and the calcium ions. This results in the formation of a stable three-dimensional network. The beads so formed were incubated in calcium chloride for 10 min after which the solution was decanted. Subsequently, this step was followed by the suspension of the crosslinked alginate into barium chloride (10 mM) for 10 mins. In order to ensure removal of excess calcium ions from bead surface, the beads were quickly rinsed in EDTA (0.05%) before coating with gelatin (1% w/v). The beads were suspended in gelatin for a period of 2 h with alternate cycles of static (10 mins) and dynamic (2 mins). To facilitate efficient reversible crosslinking of the collagen derivative, glutaraldehyde (0.4% v/v) was used and the beads were incubated in it for 20 mins. Glutaraldehyde reacts with the non-protonated F-amino groups (—NH2) of lysine or hydroxylysine through a nucleophilic addition-type reaction to yield a crosslinked gelatin coated surface. The beads were then suspended in glycine (100 mg/mL) for 40 mins to remove unreacted glutaraldehyde. In the final step, the beads were washed and suspended in calcium chloride solution (100 mM) for a period of 12 h and stored at 4° C.

The microcarriers obtained by the protocol as described herein, and the cell adhered microcarriers as described herein was evaluated by the parameters mentioned below.

### 1. Circularity Index (CI)

CI was calculated using Image J software (version 2.0.0). Briefly, oval/elliptical tool was used to fit the diameter of the beads and from the measure tool various parameters like perimeter and CI were obtained. From the perimeter value and using the formula 2πr, radius and diameter values were derived.

### 2. Cell Adherence on the Microbeads

To demonstrate cell adherence onto the fabricated Alg/Gel microbeads, 0.5×106 BM-MSCs were statically loaded onto the microbeads (50 mg) in a 24 well plate and were incubated for a period of 24 h. After the incubation period, the beads were observed under a phase contrast microscope.

### 3. Cell Seeding Protocol for Dynamic Culture

Briefly, about 30 mg of each bead type was taken and equilibrated with the media for 30 min in a spinner flask. Subsequently, each bead type was subjected to an alternate cycle of static and dynamic conditions for the first 3 h. The dynamic condition was set for 5 min (done manually for RCP and PS beads) while the static was set for 55 min and this cycle was repeated three times. Then, the microbeads were transferred to spinner flasks and maintained at a constant dynamic condition with stirring speed set to 85 rpm for 24 h. The RCP and the polystyrene beads were pooled in a single spinner flask while the sodium alginate beads were cultured separately in another spinner flask under dynamic condition. After 24 h, the beads were analysed for cell adherence and cell viability.

### 4. Live Dead Assay

Fluorescence based Live/Dead assay based on calcein-AM (Cat. No.: C1430, ThermoFisher) and ethidium homodimer (Cat. No.: 46043, Sigma-Aldrich) was used according to the manufacturers' protocol and imaged using a Laser scanning Confocal Microscope (Nikon C2 with Nis Elements 5.0 Imaging Software). Hoechst (Cat. No: 14533, Sigma Aldrich) staining was used to label nucleus. The live cells were labelled in green, dead cells in red and nuclei in blue. Maximum intensity projections of the Z stacks (spanning about 50 μm) were made using Image J software (version 2.0.0).

5. Cell Viability Testing with Trypan Blue

Cell suspension was diluted in trypan blue (Cat. No.: T8154, Sigma Aldrich) in the ratio of 1:1, and the non-viable cells (in blue) and viable cells (unstained) were counted in a Neubauer chamber to determine the cell viability index.

### 6. Immunostaining

Immunofluorescence staining stem cell markers was done using routine antibody staining protocol. Briefly, adhered cells on the beads were fixed in 10% neutral buffered formalin for 30 mins at room temperature (RT) and washed with PBS containing triton (0.1%) for 5 mins. For blocking, 1% bovine serum albumin (BSA) was used and the samples were incubated for 45 mins at RT. Primary antibody diluted in the blocking buffer was incubated overnight at 4° C. and washed with PBS (3×; 10 minutes each). Secondary antibody diluted in the blocking buffer was incubated for 1 h and washed with PBS (3×; 10 minutes each) and finally incubated with Dapi for 10 min in PBS. Samples were imaged either using a Laser scanning microscope (Nokia C2) or Keyence microscope. Maximum intensity projections of the Z stacks (spanning about 50 μm) were made using Image J software (version 2.0.0), wherever applicable.

### Decellularization Protocol & Dissolution of Alg/Gel Microbeads

Cell-laden Alg/Gel microbeads were incubated in a dissolution buffer, which is a combination of sodium chloride (0.15 M) and trisodium citrate (0.055 M) trisodium citrate, over a period of 9 minutes at room temperature. After microbead dissolution, the suspension was centrifuged and the cells were pelleted out. The cells were resuspended in PBS and a trypan blue staining assay was performed to count the number of viable cells.

### Estimated Number of Beads for Bioreactor

As the average radius of Alg/Gel beads is ˜ 200 μm, the following calculations will be helpful to arrive at the requirements to culture 10 million cells in a volume of 500 mL bioreactor that maintains constant stirring and dynamic culture conditions.

i. Micro sphere/bead radius will be ˜ 200 μm (diameter=˜400 μm)

According to sphere volume equation=(4/3 π r3), micro sphere volume equal to (3.35×107) (μm)

ii. Therefore, in 1 ml of alginate solution, the numbers of microbeads are calculated to be
a. 1 ml of solution volume equal to 1 cc=10₁₂ (μm)₃.
b. 1 ml solution contains=10₁₂ (μm)₃/vol. of each microbead=10₁₂ (μm)₃/(3.35×10₇) (μm)3=29850=˜3×10₄

Hence number of beads required for the 500 mL bioreactor=3×10₄×500=1.5×10₇.

### Preparation of Microcarriers for Bioreactor

Approximately, 200 g of the microcarriers/beads was weighed in 120 mL of PBS buffer and rehydrate.

The mixture was allowed to hydrate for at least 1 h before heat sterilization by autoclave (121° C. for 15 min).

After heat sterilization, the microcarriers/beads will settle to the bottom and was washed with 50 mL of culture medium. The washing step was repeated twice

After this procedure, microcarriers are ready to use in cell culture.

### Culturing on the Surface of Microcarriers in a 500 ml Bioreactor

The mesenchymal stem cells (MSCs) were grown in sufficient numbers in a two-dimensional (2D) xeno-free culture conditions, and then trypsinized to get a single cell suspension.

A day prior to the experiment, 500 ml spinner flasks or bioreactors was autoclaved if required. If sterile spinner flasks/bioreactors are available, they will be readily used.

The autoclaved/sterile spinner flasks were washed once with 50 mL DPBS. After that, 200 g of microcarriers suspended in 150 mL of xenofree MSCs medium was added to each of the 500 ml spinner flask or bioreactor.

Spinner Flasks or bioreactors were equilibrated for 30 min in a standard tissue culture incubator.

Following that, 10 million MSCs suspended in 50 mL volume were added to each 500 mL flask or bioreactor.

To achieve uniform cell seeding, the spinner flasks or bioreactors were placed on magnetic stirrer plate and initial stirring for 5 min will be started at 10-30 rpm for vertical impellers while 30-8 rpm for horizontal impellers, followed by rest for 55 min, at 37° C. and 5% CO₂, for a total of 1-hour static/dynamic incubation cycle. These cycles will be repeated for four times.

At the end of the seeding, 150 mL of medium was added to the culture and continuous stirring at 15-30 rpm for vertical impellers while 30-85 rpm for horizontal impellers was done.

The total volume will become 400 ml of media with beads and cells.

Half of the total medium volume was changed every day. For this, the beads were allowed to settle to the bottom of the bioreactor and carefully, 200 ml of the medium was carefully aspirated and replaced with fresh xenofree MSCs medium.

The culture was maintained up to 7-14 days.

### Results

### Size Distribution of the Microcarriers

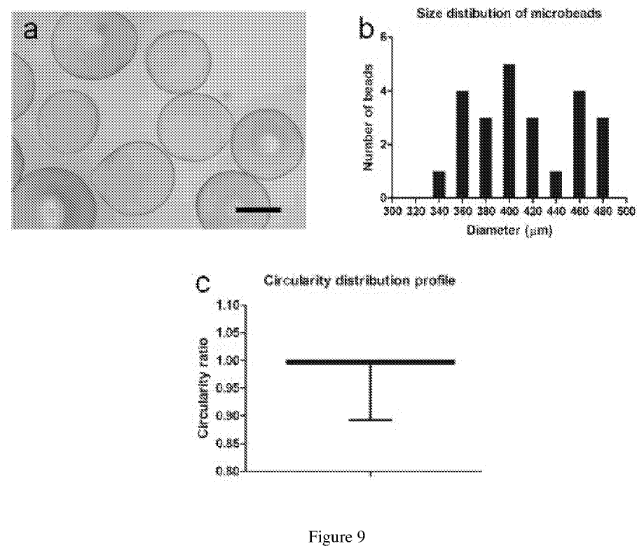

The alginate-gelatin microcarriers were obtained as mentioned previously in the present Example 6. The size of the microbeads was analyzed using the phase contrast mode of the EVOS imaging system. A batch of microbeads was assessed, and the size distribution of the alginate gelatin beads were plotted using the GraphPad Prism 5 software. In addition, the circularity profile of the microbeads was also analysed (FIG. 9). The size of the microbeads was found to be in the range of 409.84±44.14 μm while the circularity ratio of >0.90 clearly indicates that the shape of the microbeads are more or less a proper sphere (circularity ratio of 1 indicates a perfect sphere).

### Cell Adherence on the Microcarriers/Microbeads

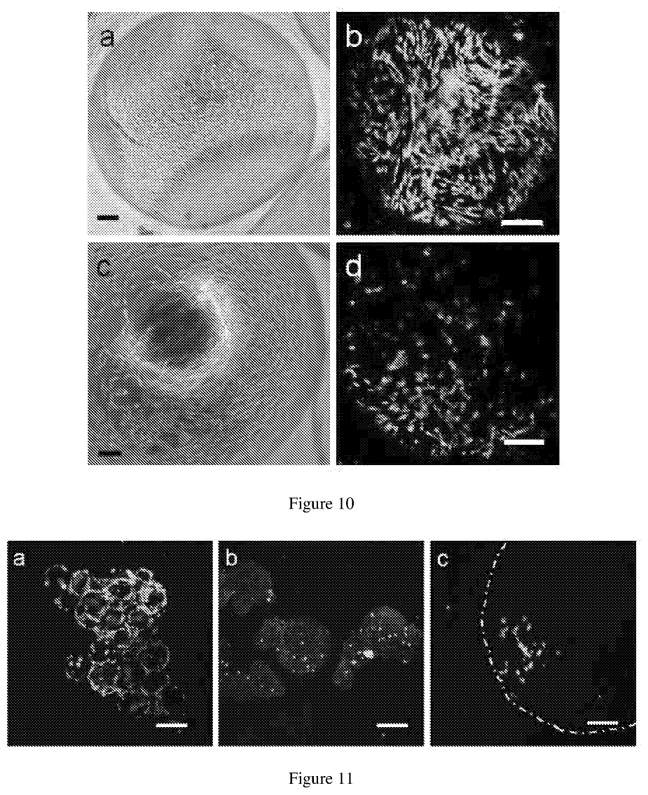

Prior to dynamic culture, microbeads were suspended in a spinner flask containing 20 mL of media and were mechanically stirred for a period of 72 h to check for their shape and integrity. The results showed that the Alg/Gel microbeads provided a microenvironment conducive for cell adhesion (FIG. 10 A). Next, to confirm the viability of cells adhered onto the microbeads, a live/dead assay was performed. Results from live/dead assay showed that a vast majority of cells on the fabricated microbeads were viable (FIG. 10 B) which convincingly demonstrates the cytocompatibility of the gelatin-coated alginate beads.

To evaluate the long-term culture of cells on the microbeads, cell-loaded microbeads were cultured under dynamic conditions for 72 h. The cells used for the present Example is obtained by culturing the BM-MSC as per the protocol as described in Example 2. The cultured BM-MSC is further used for expanding as per the microcarrier based method as described in the present Example 6. It can be contemplated that BM-MSC obtained commercially can also be used for expanding as per the present protocol.

Subsequently, microbeads were visualized under a phase contrast microscope and a live/dead assay was performed to determine cell adherence, proliferation and viability. Unsurprisingly, the engineered Alg/Gel microbeads demonstrated good stability, surface favorable for cell attachment and negligible cytotoxicity (FIGS. 10 C and 10 D).

Comparative Analysis of the Cell Culture Process Using Alg/Gel Microbeads as Disclosed in the Present Disclosure with Commercially Available Polystyrene (PS) and Recombinant Collagen Peptide (RCP) Beads

The primary purpose of the 3D microcarrier system is to facilitate the adherence of cells and their expansion in a bioreactor setup. Presently, PS and RCP beads are commercially available and have been proved to be efficient in expanding cells in a 3D dynamic culture system. Hence, the fabricated Alg/Gel microbeads as disclosed in the present disclosure were subjected to the same conditions as the other two bead types to get a comparative analysis between all three microcarrier types.

Adherence of cells—The results clearly indicate that the cells adhered significantly to the PS beads as opposed to the other two bead types (FIG. 11). Even though the number of cells that had adhered to RCP (FIG. 11 B) and Alg/Gel microcarriers (FIG. 11 C) were lesser than PS microbeads (FIG. 11 A), the viability of cells was found to be unaffected. This indicates that the components used in the preparation of the Alg/Gel beads are cytocompatible and further optimization of these beads would facilitate better adherence of the cells.

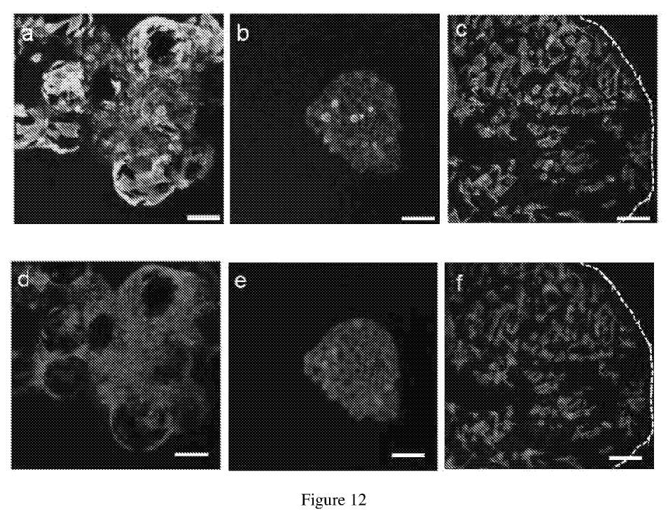

Expression of MSC stemness and stress markers—The expression of alpha smooth muscle actin (αSMA), a stress fiber marker which indicates differentiation to a myofibroblast lineage, was evaluated and compared on cells cultured on all three bead types: PS, RCP and Alg/Gel beads. The results (FIG. 12) show that compared to PS microcarriers (FIG. 12 A), RCP (FIG. 12 B) and Alg/Gel microbeads (FIG. 12 C) demonstrated weak expression of αSMA. On the other hand, PS microcarriers (FIG. 12 D) demonstrated better CD90 stem cell marker expression compared to RCP (FIG. 12 E) and Alg/Gel microcarriers (FIG. 12 F).

Decellularization via dissolution of Alg/Gel microbeads—One of the major advantage of the cell culture process using alginate-gelatin microbeads as disclosed in the present disclosure is the ease of recovery of the cultured cells as compared to the available technique in the field. The cells cultured using the microbeads as described herein are amenable to easy recovery by dissolving the microbeads by a protocol as previously described in Example 6. Whereas, such a simple recovery process is not possible by using the PS or RCP beads. In the process using PS or RCP beads, the cells are recovered by decellularization process which is time consuming and a costly affair. Also, the cell-recovery percentage is a concern.

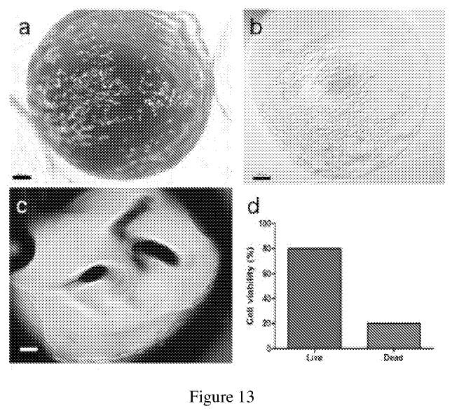

After adherence and expansion of cells on Alg/Gel microcarrier beads, the recovery of cells via minimal manipulation of microbeads and the viability of harvested cells were evaluated. The results are indicative of the fact that the beads were completely dissolved within 10 mins and the viability of the cells (˜80%) was not compromised by the dissolution buffer or by the degraded microbead products (FIG. 13).

### Comparison Matrix Between Alg/Gel Microcarriers, Polystyrene (PS) and Recombinant Collagen Peptide (RCP) Microcarriers—

Table 2 below describes the comparison matrix of the three methods.

TABLE 2

| | | Alg/Gel | | |
|---|---|---|---|---|
| | | microbeads of the | RCP | Polystyrene |
| S. No. | Parameters | present disclosure | microbeads | microbeads |
| 1. | Size distribution (dia, μm) | 340-480 | 100-400 | 125-212 |
| 2. | Bead stability in culture | ++ | +++ | +++ |
| 3. | Dynamic cell loading | ++ | +++ | +++ |
| 4. | Cell viability on beads | +++ | +++ | +++ |
| 5. | Stress biomarkers (αSMA) | low | low | high |
| 6. | Stem cell marker (CD90) | low | low | high |
| 7. | Ease of recovering cells | One-step, Easy | Moderate difficulty | Moderate |
| | | | | difficulty |
| 8. | Weight for cell culture | 1.5 | 3 | 3 |
| | (mg/ml) | | | |
| 9. | No. of microbeads/mg | 50-100 | 500-1000 | 240 |
| 10. | Total cost per gm | $10 | $1700 | $20 |

+++ excellent;
++ good;
+ fair

It can be observed from Table 2 that the microbeads of the present disclosure performs satisfactorily in terms of bead stability and dynamic cell loading. However, in terms of cell viability, expression of stress biomarker and stem cell biomarker the microbeads of the present disclosure performs better than the PS beads. Significant advantages are provides in terms of: (a) ease of cell recovery—it can be observed from Table 2, that the process of cell culturing using microbeads of the present disclosure involves an easy single step of recovering cells, whereas the other process involves moderate to high difficulty; and (b) cost—the present disclosure provides a method which is significantly economical in terms of cost as compared to the other methods.

TABLE 3

| | | | Overnight | Bead | Cell |
|---|---|---|---|---|---|
| S. No. | Components | Cross linkers | incubation | integrity | adhesion |
| 1. | Alginate (1-2%) low | Calcium chloride | Sodium | Soft bead | — |
| | viscosity + | (300 mM) | cyanoborohydride | | |
| | Gelatin (1-2%) (1:1) | | | | |
| 2. | Alginate (1-2%) medium | Calcium chloride | Sodium | Stability | No cell |
| | viscosity + | (300 mM) | cyanoborohydride | improved | adhesion |
| | Gelatin (1-2%) (1:1) | | | | |
| 3. | Alginate (1-2%) medium | Calcium chloride | Sodium | Stability | Few cells |
| | viscosity + | (300 mM) | cyanoborohydride | improved | adhered |
| | Gelatin (1-2%) (1:1) | Glutaraldehyde | | | |
| | | (0.4%) | | | |
| 4. | Alginate (1-2%) medium | Calcium chloride | Sodium | Stable upto 3 | Few cells |
| | viscosity + | (300 mM) | cyanoborohydride | days in static | adhered |
| | Gelatin (1-2%) (1:1) | Barium chloride | | | |
| | | (10 mM) | | | |
| | | Glutaraldehyde | | | |
| | | (0.4%) | | | |
| 5. | Alginate (1-2%) medium | Calcium chloride | Water | Stable in | Few cells |
| | viscosity + | (300 mM) | | static | adhered |
| | Gelatin (1-2%) (1:2) | Barium chloride | | Unstable in | |
| | | (10 mM) | | dynamic | |
| | | Glutaraldehyde | | | |
| | | (0.4%) | | | |
| 6. | Alginate (1-2%) medium | Calcium chloride | Calcium chloride | Stable in | Cells |
| | viscosity + | (300 mM) | (100 mM) | static | adhered (> |
| | Gelatin (1-2%) (1:2) + | Barium chloride | | Unstable in | 80%) onto |
| | EDTA wash for 20 sec after | (10 mM) | | dynamic | beads in |
| | crosslinking with calcium | Glutaraldehyde | | | static |
| | | (0.4%) | | | |

As per the Table 3, the first non-working example uses low viscosity alginate because of which beads are softer and no cell adhesion can be observed. The second, third, and fourth non-working examples use sodium cyanoborohydride and it was found that cell adhesion and stability is a problem. The fifth non-working example uses water and it can be observed that the beads are not stable under dynamic culture conditions. The sixth non-working example comprises an EDTA wash which was found to provide unstable beads in the dynamic culture. Therefore, the process as disclosed in the present Example is very critical for obtaining the microbeads that can be used to obtain desirable expanded population of mesenchymal stem cells.

### Example 7

### Expanding Stem Cells in Three-Dimensional (3D) Spheroid-Based Culture

### Combination of Hanging Drop and Spinner Flask Methods

The Donor-derived bone-marrow MSC were commercially procured and cultured according to the vendor's instruction.

Initially cells were thawed and cultured in 2D mono-layer in suitable culturing flasks until it reached 90% confluency.

Cells were trypsinized and counted by trypan blue staining.

Cell pellet was resuspended in an appropriate volume of media consisting of either 1:1 ratio of MSC basal media and Methyl cellulose to get 3000 cells/10 μl density or without methyl cellulose.

10 μl drops of cell suspension was added onto the lid of the 96 well plate and wells were filled with 50 μl of sterile 1×PBS for maintaining humidity

After adding the drops the lid was inverted to create hanging drop and plates were incubated at 37° C., 5% CO₂ incubator (static—hanging drop).

Within 16-24 hrs cells were aggregated and formed the spheroids

These spheroids were transferred into spinner flask with either a 1:1 ratio of MSC basal media and methyl cellulose (1%) or without methyl cellulose for dynamic culture condition and incubated at 37° C., 5% CO₂ incubator with magnetic stirring of 115 RPM (dynamic culture in spinner flask).

For control studies spheroids were cultured in MSC basal media without methyl cellulose keeping all the dynamic conditions same

Spheroids were cultured in the same condition for 5 days

Morphology and viability testing were performed by phase contrast imaging and live dead assay respectively on regular time intervals (day 3 and day 5)

On 5^{th} day spheroids were changed with EV-collect media (low serum xeno free medium) and cultured for further 48 hrs keeping all the dynamic conditions same

Morphology and viability testing were performed on 7^{th} day to assess the quality of the spheroids.

### Direct Spinner Flask Method

The Donor-derived bone-marrow MSC were commercially procured and cultured according to the vendor's instruction.

Initially cells were thawed and cultured in 2D mono-layer in suitable culturing flasks until it reached 90% confluency

Cells were trypsinized and counted by trypan blue staining

Cell pellet was resuspended in 15 ml volume of media consisting of 1:1 ratio of MSC basal media and Methyl cellulose to get 3×10⁶ cells in total volume

Cell suspension was transferred into spinner flask with either a 1:1 ratio of MSC basal media and methyl cellulose or without methyl cellulose for dynamic culture condition and incubated at 37° C., 5% CO₂ incubator with magnetic stirring of 90 RPM

For control studies cell suspension was cultured in MSC basal media without methyl cellulose keeping all the dynamic conditions same

Within 24 hrs cells were aggregated and formed the spheroids and allowed to culture in the same condition for 3 days

Morphology and viability testing were performed by phase contrast imaging and live dead assay respectively on regular time intervals

On 3^{rd} day spheroids were changed with low serum xenofree media and cultured for further 48 hrs keeping all the dynamic conditions same.

Morphology and viability testing were performed on 5^{th} day to assess the quality of the spheroids

### Evaluation of Hollow Fiber Bioreactors for the Scale Up Culture of MSCs and Exosome Production

The Hollow fiber bioreactors (HFBs) are a 3D culture system that consist of fibers fixed on a module with cells cultured on the outer surface of porous fibers. The media is then circulated through the fiber capillary lumen, mimicking the in vivo-like circulation of nutrients through blood capillaries. This type of cell culture system allows controlled shear to be applied to cells in culture with dynamic transfer of nutrients and removal of waste products. This creates a versatile cell culture system in which high cell densities can be easily achieved.

A Quantum Cell Expansion System® (Terumo BCT, Colorado, USA) can be used as a part of the present disclosure.

The surface of the hollow fibers is to be coated with human fibronectin (0.05 mg/ml) 18 hours prior to seeding cells, to promote cell adhesion.

The xenofree culture medium is to be equilibrated with a gas mixture (5% 02, 5% CO₂ and 90% N2) to provide adequate aeration.

Cells to be seeded at a density of 30×10⁶ cells, (1000 cells/cm²) in the intracapillary space (ICS) for cell adhesion for 24 hours. The cells are to be constantly fed through a continuous flow of culture medium in the extra-capillary space (ECS) with passive removal to waste.

Cells are to be harvested with trypsin as described when a confluency of >90% is reached.

For exosome production, the media is to be replaced entirely with low serum xenofree media (Rooster Bio inc.) and cells is to be cultured for 72 hours. The conditioned media will be collected and harvested as described in the present disclosure.

### Results

### Combination of Hanging Drop & Spinner Flask Methods

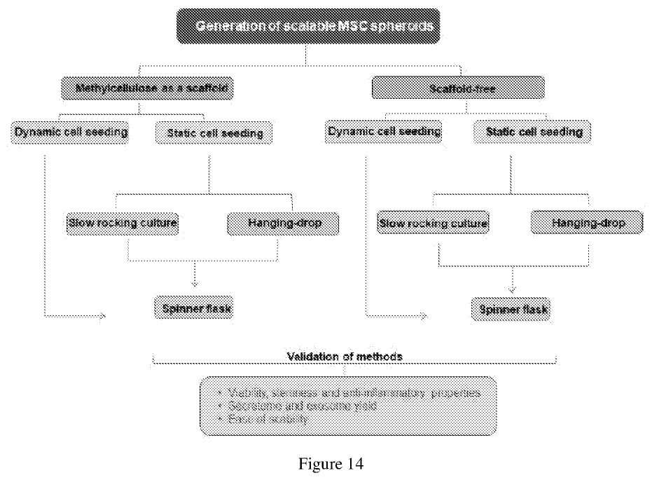

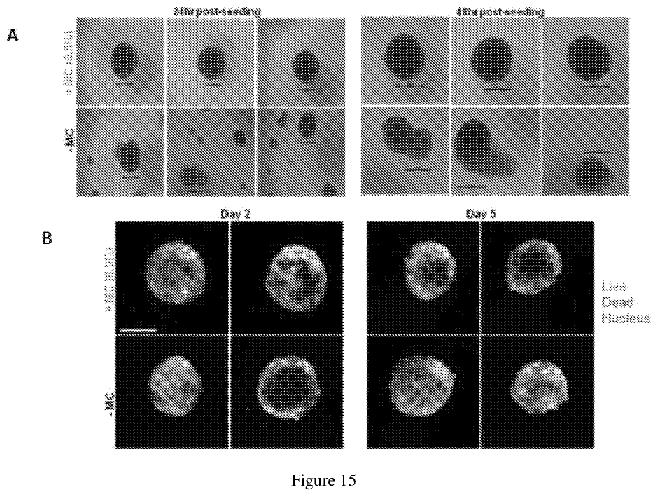

hBMMSC form compact spheroids in the presence of methyl cellulose—A scheme for the production of 3D hBM-MSC spheroids (FIG. 14) and dynamic culture for secretome and exosome production has been disclosed herein. The present data is obtained by culturing BM-MSC. The initial culturing of BM-MSC was done by the protocol explained in Example 2 and the further expansion was done by the present Example. Methyl cellulose was used to enhance the spheroid formation during the hanging drop culture. It was observed that the presence of methyl cellulose enhanced the spheroid forming capacity as evidenced by the single compact cluster of cells, whereas multiple clusters were observed in the hanging drop without methyl cellulose (FIG. 15 A). The average size of each spheroid reached up to 200 μm and was maintained throughout the culture period. The spheroids without methylcellulose showed multiple clusters of cells even after 48 h post seeding. As shown in FIG. 15B, viability staining performed on spheroids collected on day 2 and day 5 did not show a significant difference in the viability of cells in the presence of methyl cellulose when compared with the spheroids without methylcellulose. These results suggest that presence of methyl cellulose in the hanging drops reduces the spheroid forming time without affecting the viability of the cells, possibly due to the increased viscosity of the culture medium with the methyl cellulose.

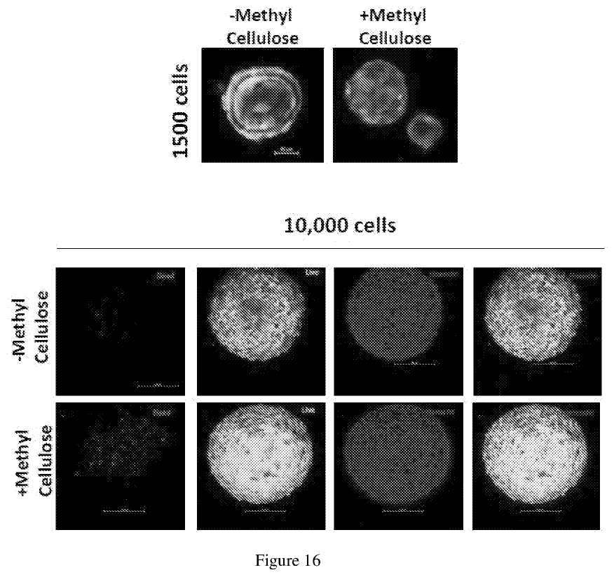

Spheroid formation at a lesser cell density of 1500 cells and higher cell density of 10,000 cells per spheroid using the hanging drop method was also demonstrated. It was found that 1500 cells produced smaller spheroids (50-100 μm) (FIG. 16 A), comparable sizes in the presence and absence of methyl cellulose while seeding at a higher density of 10,000 cells resulted in the formation of spheroids of approximately 200 μm in the absence of methyl cellulose and 200-300 μm in the presence of methyl cellulose in 24-72 hours (FIG. 16 B). As also shown in FIG. 16 B, increased cytotoxicity (dead cells) at this seeding density was also observed. Interestingly, increased cytotoxicity in spheroids plus methyl cellulose was observed compared to the spheroids without methyl cellulose (FIG. 15-16). Hence, a range is provided for the concentration of methyl cellulose that can be used in this protocol in Table 4.

TABLE 4

| SI. no | Parameters | Working range |
|---|---|---|
| 1 | Initial seeding density (cells/spheroid) | 600-10,000 |
| 2 | Conc. of Methylcellulose | 0.2-2% |
| 3 | Cell aggregation time in hanging drop | 6 h-24 h |
| 4 | Spheroid maturation time in dynamic culture | 3-7 days |
| 5 | Time window for exosome collection (post- | Day 3-Day 7 |
| | maturation) | |
| 6 | Diameter of the spheroids | Hanging drop: 100-300 μm |
| | | Direct spinner flask: 30-250 μm |
| 7 | Speed of the magnetic stirrer | 50-150 rpm |

Combination of Slow Rocking Culture Step with Spinner Flask Dynamic Culture of Spheroids

An alternate hanging drop protocol can be adopted in which the spheroid formation+/−methyl cellulose occurs on a rocking platform instead of in a hanging drop. The critical step (when compared to the technique known in the art) would be the presence of methyl cellulose in the culture medium to allow compact and rapid spheroid formation.

A 1-4 tier, multi-shelf rocker system can be placed inside an incubator at 37° C. during spheroid production. The spheroids will have continuous supply of 95% oxygen, 5% carbon dioxide gas mixture. The culture will be maintained at a rocking speed of 10-30 cycles/min with a 5-10° range of motion. Spheroids will be allowed to form at the same seeding density described in Table 4 in the presence of methyl cellulose.

hBM-MSC spheroids shows enhanced protein secretion in the dynamic culture—To address the challenges faced on obtaining the sufficient number of exosomes produced using the conventional monolayer culture; the efficiency of MSC spheroids in terms of production of quality and quantity of secretome, which includes some of the therapeutically important factors such as HGF, NGF, etc was evaluated.

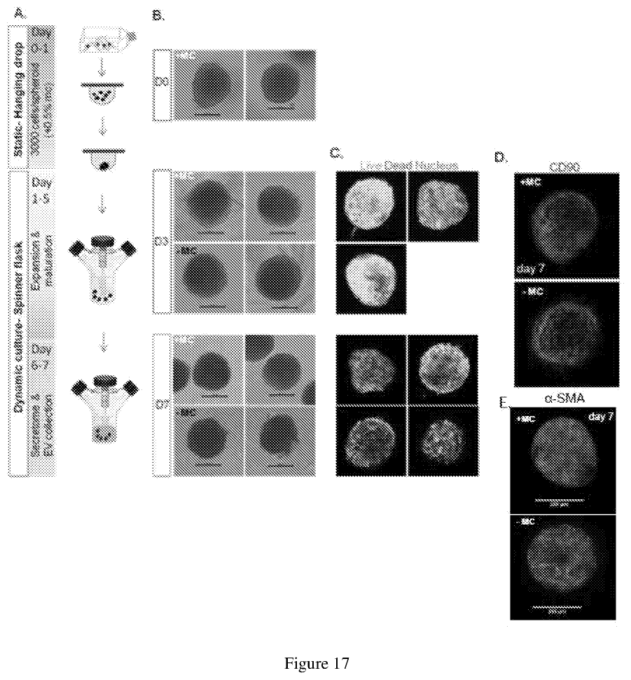

After forming the compact 3D spheroids of hBM-MSC by hanging drop culture, the spheroids were introduced into the dynamic system using spinner flask with and without methyl cellulose. FIG. 17 A, depicts the scheme of the experiment whereby spheroids formed by the static hanging-drop culture in the presence of 0.5% methyl cellulose and having a density of 3000 cells per spheroid were introduced into the dynamic culture for secretome or exosome production.

A control culture was kept without the presence of methyl cellulose in the dynamic culture system. Consistent and compact spheroids were observed in the dynamic culture throughout the culture period in both with and without methyl cellulose (FIG. 17 B). Live-dead staining performed on the spheroids from day 3 and day 7 showed a significant number of viable cells (FIG. 17 C). The expression of CD90 (stemness marker) (FIG. 17 D) and α-SMA (stress marker) (FIG. 17 E) pattern was checked after 7 days in the dynamic culture. It was observed that the CD90 expression was maintained in the dynamic culture indicating that MSC maintained their stem cell properties while low expression of α-SMA was detected in the spheroids.

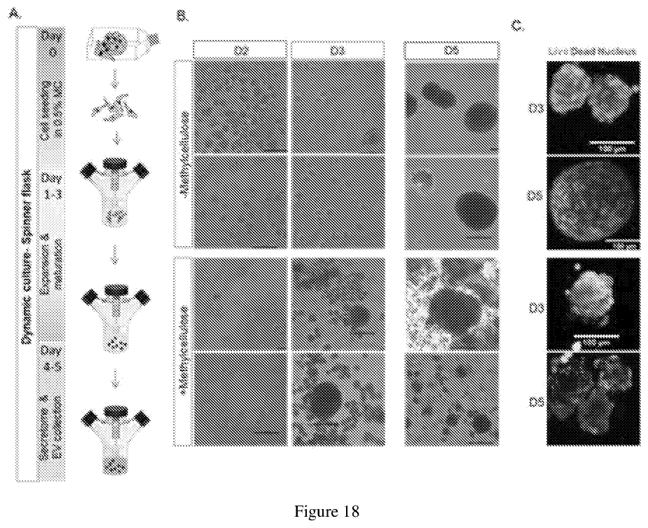

Direct spinner flask method—Besides all the efforts in scaling up MSC culture for cell and exosome therapy. There is also a growing interest in enhancing their therapeutic potential by providing the 3D culture conditions. In this regard, the use of bioreactors such as spinner flasks, rotating wall vessels and hollow fiber bioreactors have been utilized to provide a dynamic culture conditions that will increase the oxygen and nutrients supply to cells and the removal of waste products and produce fluid shear stress, which confer biomechanical cues that are the important aspect of the cellular environment and can alter the properties and behavior of cells. In this alternative method, we demonstrate the direct 3D spheroid culture by seeding the cells with a polymer-based support in a spinner flask. Unlike the published methods of spheroid generation, that require very high density of cells (˜1 million cells per ml), our method requires 5-fold lesser cell density (0.2 million cells/ml) for the spheroid formation. When cultured with 0.75% methyl cellulose, the cell formed predominantly uniform clusters ranging from 30-100 μm (FIG. 18 B). We found these clusters were stable and showed viable cells during the culture period (FIG. 18 C). In the absence of methyl cellulose, the spheroids were observed in the spinner flask, however the efficiency of the cell aggregation was lower as evidenced by the settlement of cells to the bottom of flask. Moreover, at day 5 more number of cell aggregates was observed in the spheroid cultured with methyl cellulose compared with the spheroids cultured without methylcellulose.

### Example 8

Isolation and Purification of Secretome and Exosomes from the Cell Culture

The conditioned medium was collected from the CSSC and hBMMSC according to the process as described in Example 1 and 2, respectively. The obtained conditioned medium was directly used as secretome or subjected to ultracentrifugation for isolating exosomes. Isolation of exosome from secretome was done by using three methods: (i) Single step ultracentrifugation; (ii) Sucrose based cushion density ultracentrifugation and (iii) Iodixanol density gradient ultracentrifugation. All of the three methods followed a second round of purification using size exclusion chromatography (using Captocore 700 column). Capto Core 700 is composed of a ligand-activated core and inactive shell. The inactive shell excludes large molecules (cut off˜Mr 700 000) from entering the core through the pores of the shell. These larger molecules are collected in the column flow through while smaller impurities bind to the internalized ligands. Furthermore, the resin Captocore700 is scalable to a capacity in litres.

The detailed process of each purification method is explained below:

(I) Single-Step Ultracentrifugation:

The following steps were followed to purify the exosomes using single-step centrifugation:
(i) Once the cells reached 80-90% confluency, the media was removed, and cells were washed in 1× Phosphate-Buffered-Saline (PBS) (20 ml). PBS was discarded and 260 mL of low serum xenofree media was added to the flasks and the flasks were then incubated for 72 h at 37° C., 5% CO₂.
(ii) The supernatant was collected and immediately proceeded with the pre-processing steps as described below:
   The media was centrifuged at 300×g for 10 min at 4° C., and the supernatant was collected.
   The supernatant was centrifuged at 3000×g for 20 min at 4° C. and the supernatant was collected.
   The supernatant was centrifuged at 13000×g for 30 min at 4° C. and the supernatant was collected.
   The media was then filtered through a 0.45-micron filter.
   The media was further filtered through a 0.22-micron filter.
(iii) The conditioned media was stored at 4° C. for short term storage (24 h) or at a temperature of −80° C. for long term storage (1 month).
(iv) Enrichment of exosomes pellet by ultracentrifugation: To process the cells immediately, following processing steps were followed. In case of frozen cells, the conditioned media was thawed at 4° C. prior to execution of the steps described below:
   The conditioned media was centrifuged at 100,000×g for 90 min at 4° C.
   The supernatant was removed carefully, and a clear pellet was observed at the bottom of the tube.
   The final centrifugation was done by dissolving the pellet in either PBS, or Plasma-Lyte A, or Saline. About 0.5 m of crude exosomes were stored at −80° C. for QC.

(II) Sucrose-Based Cushion Density Ultracentrifugation:

The following steps were followed to purify the exosomes using sucrose-based cushion density centrifugation:
(i) Once the cells reached 80-90% confluency, the media was removed, and the cells were washed in 1× Phosphate-Buffered-Saline (PBS) (20 ml). PBS was discarded and 260 mL of low serum xenofree media was added to the flasks and the flasks were then incubated for 72 h at 37° C., 5% CO₂.
(ii) The supernatant was collected and immediately proceeded with the pre-processing steps as described below:
   The media was centrifuged at 300×g for 10 min at 4° C., and the supernatant was collected.
   The supernatant was centrifuged at 3000×g for 20 min at 4° C. and the supernatant was collected.
   The supernatant was centrifuged at 13000×g for 30 min at 4° C. and the supernatant was collected.
   The media was then filtered through a 0.45-micron filter.
   The media was further filtered through a 0.22-micron filter.
(iii) The conditioned media was stored at 4° C. for short term storage (24 h) or at a temperature of −80° C. for long term storage (1 month).
(iv) Enrichment of exosomes pellet by ultracentrifugation: To process the cells immediately, following processing steps were followed. In case of frozen cells, the conditioned media was thawed at 4° C. prior to execution of the steps described below:
(v) The conditioned media was centrifuged at 100,000×g for 90 min at 4° C.
(vi) The supernatant was removed carefully, and a clear pellet was observed at the bottom of the tube.
(vii) Purification of the enriched exosomes by 30% sucrose density ultracentrifugation:
   The enriched exosomes were transferred on to 30% sucrose (1M) containing ultracentrifuge tube (according to the process as described in Gupta, S., Rawat, S., Arora, V. et al. An improvised one-step sucrose cushion ultracentrifugation method for exosome isolation from culture supernatants of mesenchymal stem cells. Stem Cell Res Ther 9, 180 (2018). https://doi.org/10.1186/s13287-018-0923-0).
   The ultracentrifuge tube was spun at a speed of 1000000 g for 2 hr at 40 C, and the acceleration and deceleration were set to zero.
   The supernatant was carefully removed, and the exosomes were resuspended in sterile 1×PBS, in order to remove the sucrose and to obtain the exosomes in pellet.
   The exosomes were aliquoted and were stored at store at −80° C.

(III) Iodixanol Density Gradient Ultracentrifugation:

The following steps were followed to purify the exosomes using iodixanol cushion density centrifugation:
(i) Once the cells reached 80-90% confluency, the media was removed, and the cells were washed in 1× Phosphate-Buffered-Saline (PBS) (20 ml). PBS was discarded and 260 mL of low serum xenofree media was added to the flasks and the flasks were then incubated for 72 h at 37° C., 5% CO₂.
(ii) The supernatant was collected and immediately proceeded with the pre-processing steps as described below:
   The media was centrifuged at 300×g for 10 min at 4° C., and the supernatant was collected.
   The supernatant was centrifuged at 3000×g for 20 min at 4° C. and the supernatant was collected.
   The supernatant was centrifuged at 13000×g for 30 min at 4° C. and the supernatant was collected.
   The media was then filtered through a 0.45-micron filter.
   The media was further filtered through a 0.22-micron filter.
(iii) The conditioned media was stored at 4° C. for short term storage (24 h) or at a temperature of −80° C. for long term storage (1 month).
(iv) Enrichment of exosomes pellet by ultracentrifugation: To process the cells immediately, following processing steps were followed. In case of frozen cells, the conditioned media was thawed at 4° C. prior to execution of the steps described below:
   The conditioned media was centrifuged at 100,000×g for 90 min at 4° C.
   The supernatant was removed carefully, and a clear pellet was observed at the bottom of the tube.
   The pellet was dissolved in 36 mL low serum xenofree media (36 mL per 300 mL starting conditioned media). About 0.5 m of crude exosomes were stored at −80° C. for QC.
(v) Density gradient ultracentrifugation (DGUC):
   An Iodaxinol (IDX) gradient fractions were prepared by floating 3 ml of 10% w/v IDX solution ((Sigma #D1556) containing NaCl (150 mM) and 25 mM Tris:HCl (pH 7.4), over 3 ml of 55% w/v IDX solution.
   The conditioned Media (6 ml) was floated on the top of the IDX cushion and was then allowed to ultracentrifuge by using a Beckman Coulter SW 40 Ti rotor for 4.5 hours at 100,000×g (4° C.).
   Twelve IDX gradient fractions (1 ml each) were collected from the top of the gradient. Fraction collection was carried out on ice and each fraction was collected into pre-chilled 1.5 ml tubes.
   About 9 IDX gradient fractions were transferred into a fresh ultracentrifuge tube and 11 ml PBS was added to the 1 mL fraction. The ultracentrifugation step was repeated at 100,000×g for 4 h in Optima XPN-100 ultracentrifuge using a Beckman Coulter SW 40 Ti rotor at 4° C.
   The supernatant was discarded. The exosome pellet was then resuspended in 1 ml PBS.
   About 50-100 μL aliquots of exosomes were stored at 4° C. for short term (2-3 days) and −80° C. for long term storage.

(IV) Purification of Exosomes by Size Exclusion Chromatography Using CaptoCore 700 Column:

Exosomes isolated by the above three methods (I, III, and III) were further purified by running through a size exclusion chromatography column—1 ml (CaptoCore 700, GE). The steps are described below:
(v) A 1 ml column was equilibrated with 1×PBS (5 times). Post equilibration, the exosome sample (50-100 μl) was loaded into the column.
(vi) The exosomes were eluted in a total of 1 mL 1×PBS/PlasmaLyte A/Saline in fractions of 35 μL (approximately 26 fractions). About 2-3 fractions contained exosomes. The fractions were then manually collected into 1.5 ml tubes kept on ice.

The tubes containing purified fractions of exosomes were stored at 4° C. for short term (2-3 days) and −80° C. for long term storage.

### Results

Characterization of the secretome obtained from the conditioned medium collected by the culture methods as described in the Examples 3 and 4.

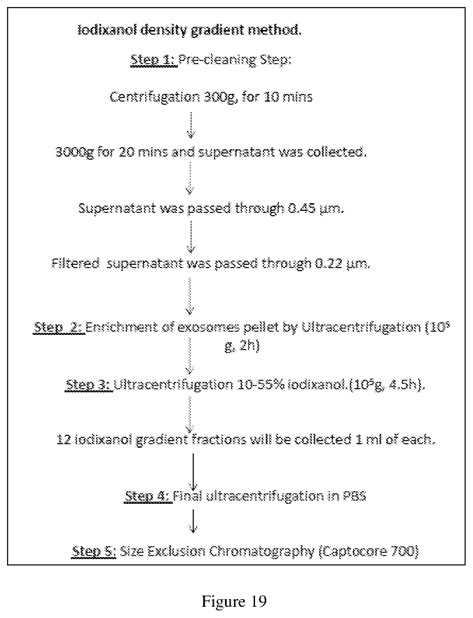

The conditioned medium was collected from the CSSC and hBMMSC 2D cultures as described above. The obtained conditioned medium was directly used as secretome or subjected to ultracentrifugation for isolating exosomes. Isolation of exosome from secretome was done using Iodixanol density gradient ultracentrifugation (FIG. 19). The purified exosomes were further characterized using multiple methods like the Nano tracking analysis (NTA), transmission electron microscopy (TEM) and western blot.

Characterization of Secretome from BM-MSC and CSSC Cultured by Two-Dimensional Culture Methods

The respective cells were obtained by the methods as described in Example 2 and 1, respectively.

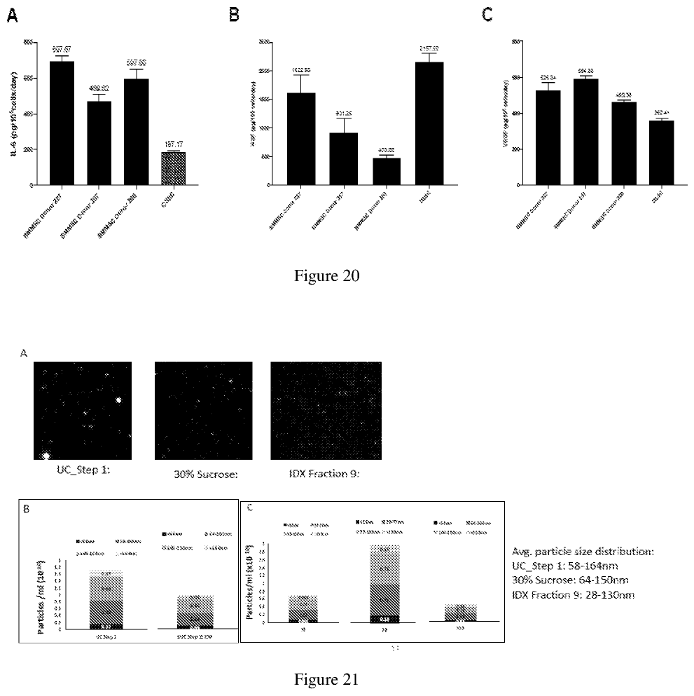

The secretome of BMMSCs from three independent donors (#200, #227, #257) were harvested alongside CSSCs and secreted levels of VEGF, HGF and IL-6 were quantified. CSSCs were found to secrete significantly lower levels of pro-inflammatory IL-6 compared to BMMSCs while priming of BMMSCs with CSSC-conditioned medium resulted in a marked decrease in the level of IL-6 secreted by the primed BMMSCs (FIG. 20 A, and FIG. 6 B). BMMSCs from all three donors were found to secrete more VEGF than CSSCs (FIG. 20 C), while CSSCs were observed to express more HGF levels (FIG. 20 B).

### BMMSCs Cultured in 3D as Spheroids as Compared to 2D Culturing Methods

The MSC (hBM-MSC) were cultured as per the method described in the Example 7 for 3D spheroid-based culturing, and as per the Example 2 for 2D based culturing.

The protein content in the secretome obtained from the conditioned medium in 3D spheroids and 2D methods was quantified by Bradford method. The amount of protein was normalised to per millions of cells and presented as protein yield per million cells per day. A differential amount of protein was found to be present in the secretome of 2D and 3D samples. When compared with 2D hBM-MSC, which were incubated in secretome collection medium, a 4.8-folds and 3.2-folds more protein in 3D spheroids cultured with and without methyl cellulose respectively, was observed. The increase in the protein content may directly correlate with the amount of therapeutically important factors present in the secretome (Table 5). Table 6 depicts the cell viability, biomarker expression levels, and total secreted protein. Thus, it can be inferred that 3D culturing methods as described in the Examples 6 and 7 are a viable option to scale-up MSC-exosome production in order to meet the current challenges faced in obtaining therapeutic dose of exosome which is cost-effective, consistent and less labor intensive.

TABLE 5

| | | | Fold increase in |
|---|---|---|---|
| | Cell Number | Total protein secreted/ | protein compared |
| Cells Types | (million) | million cells (mg)/day | to 2D |
| 2D hBM-MSC | 6.5 | 0.045 | — |
| 3D hBM-MSC Hanging drop + | 125 spheroids (0.375) | 0.217 | 4.8 |
| Spinner flask (+methyl | | | |
| cellulose) | | | |
| 3D hBM-MSC Hanging drop + | 125 spheroids (0.375) | 0.145 | 3.2 |
| Spinner flask | | | |
| (−methyl cellulose) | | | |

TABLE 6

| | | 2D cultures (as | Microcarrier culture | Spheroid culture (as |
|---|---|---|---|---|
| | | per Example 2; | (as per Example 6; | per Example 7; |
| S. No. | Parameters | hBM-MSC) | hBM-MSC) | hBM-MSC) |
| 1. | Cell viability | >98% | >90% | >90% |
| 2 | Biomarker | High expression of | Moderate expression of | High expression of |
| | expression | CD90 | CD90 | CD90 |
| 3. | Total secreted | 0.045 | — | 0.145-0.217 |
| | protein (mg/million | | | (3- to 5-fold increase) |
| | cells/day | | | |

Characterization of Purified Exosomes from MSCs (2D Culture):

The conditioned medium was collected from the CSSC and hBMMSC 2D cultures as described above (Example 1 and 2, respectively). The obtained conditioned medium was directly used as secretome or subjected to ultracentrifugation for isolating exosomes. Isolation of exosome from secretome was done using three methods namely (i) Single step ultracentrifugation; (ii) Sucrose based cushion density ultracentrifugation and (iii) Iodixanol density gradient ultracentrifugation. The three protocols will be followed by a second round of purification using size exclusion chromatography (CAPTOCORE 700).

The purity of exosomes isolated by the methods is the key differentiating factor between the protocols: Iodixanol protocol (highest purity)>30% sucrose cushion>single step ultracentrifugation (lowest purity) (see FIG. 21). Comparison of exosome population isolated by Single step ultracentrifugation (UC_Step 1), 30% sucrose cushion and iodixanol gradient ultracentrifugation protocols: (A-C) demonstrate the heterogeneity of the exosome particle size obtained in each method of purification. Single step UC purification of exosomes results in isolation of particles in the range of 50-170 nm, 30% sucrose cushion gives us particles in the range of 60-150 nm while iodixanol gives us a tighter range of 30-130 nm. Particle heterogeneity: Single step UC>30% sucrose cushion>iodixanol gradient method. UC Step 1: single step ultracentrifugation; SUC Step 2:CM: 30% Sucrose cushion ultracentrifugation; IDX F8-F10: Iodixanol density gradient ultracentrifugation fractions 8, 9, 10.

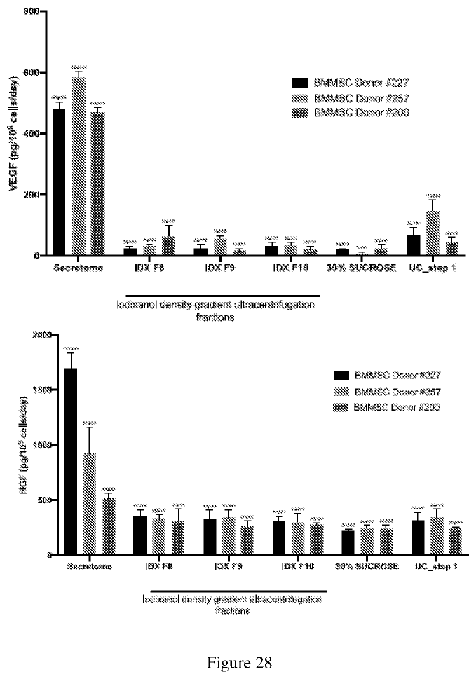

Capto Core 700 is composed of a ligand-activated core and inactive shell. The inactive shell excludes large molecules (cut off˜Mr 700 000) from entering the core through the pores of the shell. These larger molecules are collected in the column flow through while smaller impurities bind to the internalized ligands. Furthermore, the resin Captocore700 is scalable to a capacity in litres. Exosomes of different purities will be developed for target indication specificity. For example, a combination of iodixanol density gradient Ultracentrifugation or 30% sucrose cushion+Captocore700 would give the highest purity with minimal contamination with angiogenic factors (e.g. VEGF) that would be ideal for application in avascular tissues such as cornea (FIG. 28). A less rigorous purification protocol such as 30% sucrose or iodixanol density gradient ultracentrifugation only protocol would be useful in the treatment of vascular tissue related diseases where the presence of angiogenic factors would not bear any adverse effects e.g. ARDS (lung).

The purified exosomes were further characterized using multiple methods like the Nano tracking analysis (NTA), transmission electron microscopy (TEM), western blot and ELISA based immune assays.

### Working Example 1: Characterization of Purified Exosomes from BMMSCs by Iodixanol Gradient Ultracentrifugation

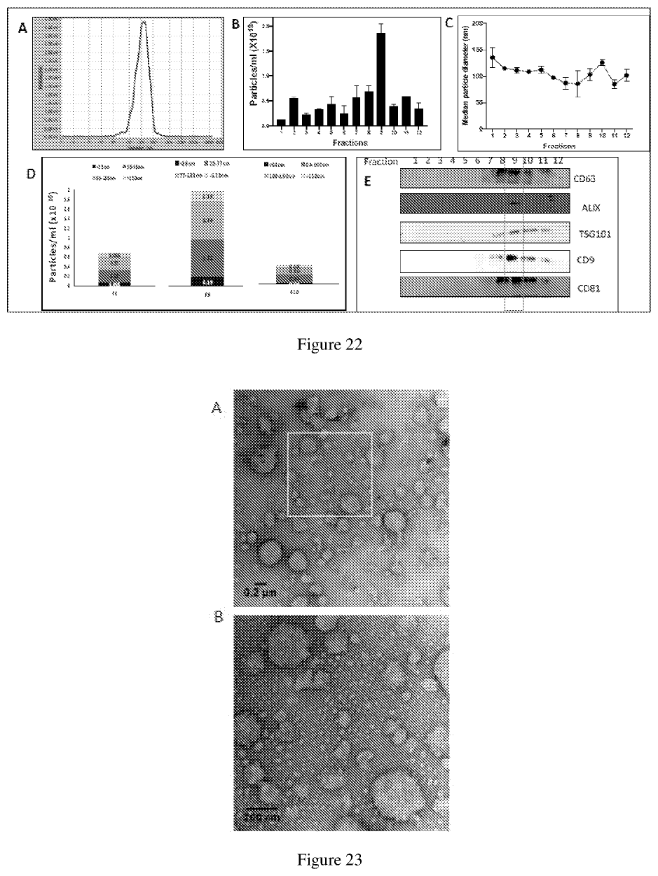

Characterization of hBM-MSC derived exosomes: Conditioned media was processed by density gradient ultracentrifugation. A total of 12 fractions were collected and characterized by nanoparticle tracking analysis (NTA, quantitative) and western blot (qualitative) (FIG. 22).

FIG. 22 A-B depicts the particle concentration of fraction 9 (F9): 1.8×10¹⁰/ml); C. Median particle diameter in nm ranged between 100-150 nm; D. Avg. size distribution of F9: 28-133 nm. Particle size distribution and particle number were determined by NTA. Particles were detected at 11 different positions of the cell and averaged. Each sample was run in 3 technical replicates. E. Exosomes (fraction 9) isolated from hBM-MSCs were positive for typical exosome markers including CD63, CD9, CD81, ALIX and TSG101.

FIG. 23 depicts the Transmission Electron Microscopy (TEM) images of exosomes isolated by iodixanol density gradient ultracentrifugation. Lower magnification of representative images is shown in (A) and the respective magnified image (marked in yellow box) is shown in (B). Scale bars (0.2 um (E), and 200 nm (F)). The TEM images shows exosomes in the expected size range of about 150-250 nm range and complements the NTA data.

### Working Example 2: Characterization of Purified Exosomes from BMMSCs Purified by a Combination of Iodixanol Gradient Ultracentrifugation and Size Exclusion Chromatography

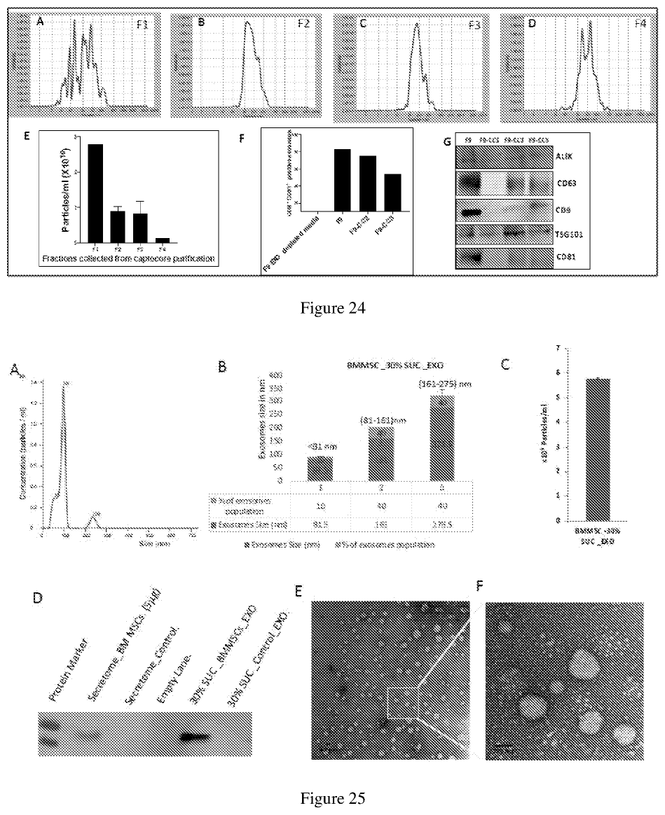

Purification of exosomes by size exclusion chromatography: Column was equilibrated with PBS 5 times. The sample (100 ul of F9) was loaded and eluted in 1 ml of PBS (as per reference) in 35 μl fractions (26 fractions). Eluted subfractions 2 & 3 were found to contain maximum yield of exosomes. The exosome profile, size distribution and protein cargo were also characterized (FIG. 24). The fractions 2&3 were pooled and tested in functional assays (hereafter referred as F9-CC). FIG. 24 depicts the Exosome size distribution and cargo characterization post size exclusion chromatography. (A-D) All fractions up to F7 were run on NTA. From F5, no particles were detected and only alternate fractions were run thereon. (E) Particle concentration per fraction (Fraction 9 was diluted into two fractions (2+3). (F) Flow cytometry analysis of fraction 2 and 3 from captocore purification identified 75% and 54% of the exosome population in fraction 2 and fraction 3 to be CD81/CD9 positive, respectively. (G) Western blot analysis of exosome markers CD81, CD9, CD63, ALIX and TSG101 in captocore purified fraction 9.

### Working Example 3: Characterization of Purified Exosomes from BMMSCs Purified 30% Sucrose Cushion Ultracentrifugation

The 30% sucrose cushion density ultracentrifugation yielded higher particle numbers compared to iodixanol (approximately 5 folds higher) (FIG. 25 A-C). However, the particle size distribution was more heterogenous with roughly 40% of the exosomes falling in the size range of >150 nm (161-275 nm) (FIG. 25 B).

The exosomes were found to express CD9, a key exosome marker and maintained their integrity/morphology in solution as shown in FIGS. 25 D and E-F respectively. FIG. 25 depicts (A-C) Size distribution analysis of exosomes purified from BMMSCs by 30% cushion-based sucrose density method using Nano Tracking Analysis (NTA). A representative image of histogram is shown in A. The averaged data from 3 independent readings of size distribution are presented in B. (C) The total yield of exosomes from 30% sucrose cushion ultracentrifugation determined by NTA. (D). Western blot analysis for exosome marker CD9. Protein samples from secretome and exosome preparation were separated on a 12% SDS PAGE gel and antibody against CD9 was used to identify exosomes. CD9 was present both in secretome and exosome samples showing expected size of 24-27 Kda and the control samples were negative. (E and F) Transmission Electron Microscopy (TEM) images of exosomes isolated by 30% sucrose method. Lower magnification of representative images is shown in (E) and the respective magnified image (marked in yellow box) is shown in (F). Scale bars (0.2 um (E), and 200 nm (F)). The TEM images shows exosomes in the expected size range of about 150-250 nm range and complements the NTA data.

### Working Example 4: Characterization of Purified Exosomes from CSSCs Purified by Sucrose Cushion Ultracentrifugation and Iodixanol Density Gradient Ultracentrifugation

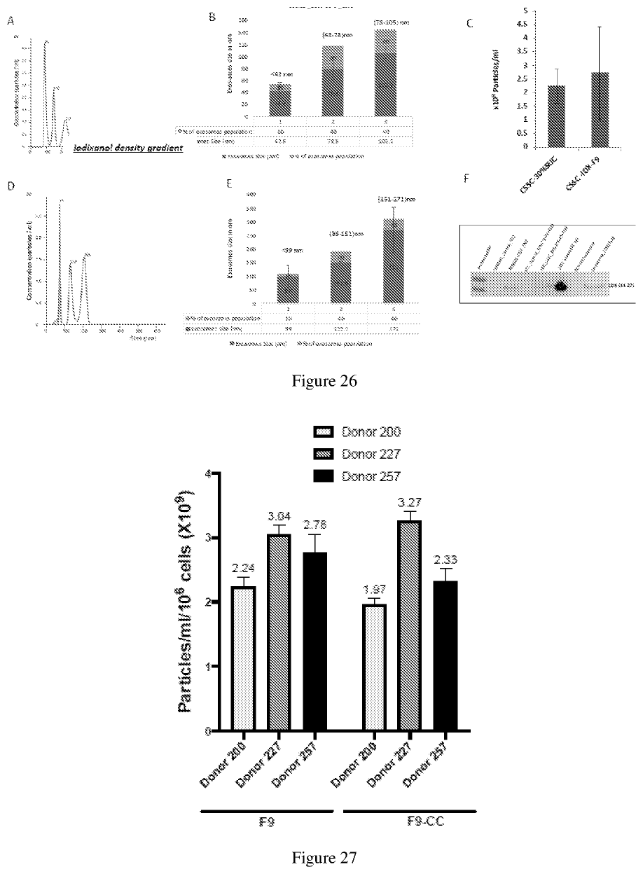

Exosomes from CSSCs isolated by both 30% sucrose cushion (FIG. 26 A-B) and Iodixanol density gradient ultracentrifugation (FIG. 26 D-E) were more heterogenous compared to BMMSC derived exosomes. However, the particle numbers isolated by iodixanol gradient was comparable to the exosome yield from BMMSCs (FIG. 26 C). The exosomes isolated by both methods expressed similar levels of exosomal marker CD9 (FIG. 26 F). FIG. 26 depicts (A-C) Size distribution analysis of exosomes purified from CSSCs by 30% sucrose cushion density (30% SUC) based ultracentrifugation and (D-E) iodixanol density gradient ultracentrifugation (IDX Fraction 9 (IDX-F9)) method using Nano Tracking Analysis (NTA). A representative image of histogram is shown in A, D for 30% SUC and IDX-F9 respectively. The averaged data from 3 independent readings of size distribution are presented in B &E for 30% SUC and IDX-F9 respectively. (C) The total yield of exosomes from 30% SUC and IDX-F9 respectively determined by NTA. (F) Western blot analysis for exosome marker CD9 for 30% SUC and IDX-F9. Protein samples from secretome and exosome preparation were separated on a 12% SDS PAGE gel and antibody against CD9 was used to identify exosomes. CD9 was present both in secretome and exosome samples showing expected size of 24-27 Kda and the control samples were negative.

### Reproducibility of the Protocol of Exosome Production (2D Culture) of the Present Disclosure:

Three independent donors of hBMMSCs were expanded using the 2D protocol described above. Cells were expanded in xenofree culture medium and exosomes were collected post 72 h incubation in RoosterBio Low serum xenofree media. Exosomes were purified by Iodixanol density gradient ultracentrifugation from a total volume of 200 ml per donor. Fraction 9 was collected (F9) and half of the fraction was further purified by size exclusion chromatography (F9-CC). With the present protocol of the disclosure an average of 2.7×109±0.24 particles per 1 million BMMSCs (n=3 donors) (FIG. 27) were purified. This confirms the reproducible production of a high yield of exosomes from cells from different donors using our protocol. Thus, it was confirmed that the protocols described in this example section can be employed for all cell types listed in the present disclosure. FIG. 28 depicts the comparison of purity of exosomes purified by three methods (i) single step ultracentrifugation (UC_step 1), (ii) s30% sucrose cushion (iii) iodixanol gradient UC (IDX). (A) Sucrose cushion and iodixanol gradient methods gave comparable purity and low levels of VEGF compared to UC_Step 1 (single step ultracentrifugation) while retaining therapeutic factors such as HGF (B).

### ADVANTAGES OF THE PRESENT DISCLOSURE

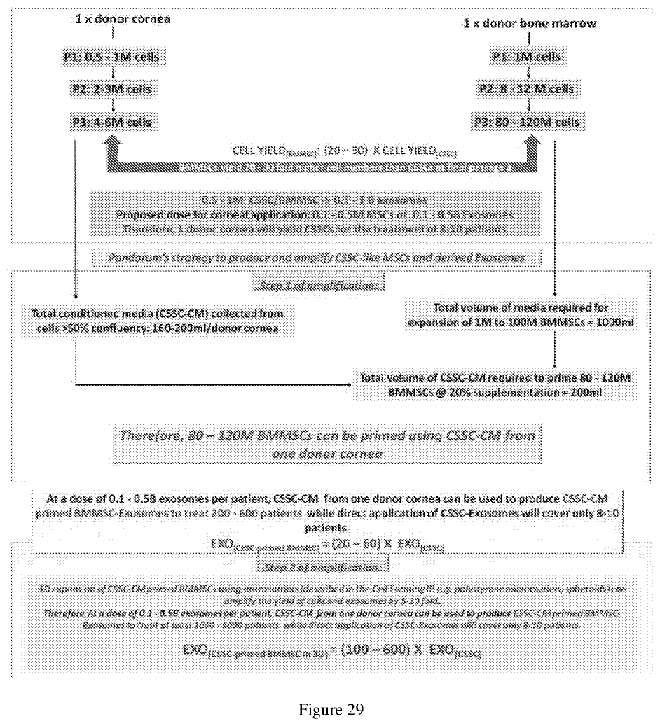

FIG. 29 depicts the comparison of scalability of CSSC-CM primed MSC versus CSSC in clinical applications. Priming hBM-MSCs with CSSC-CM skews the phenotype of BM-MSCs towards a more CSSC-like profile. This will help in circumventing the need to isolate fresh CSSCs from human donor corneas, which are difficult to procure and will also minimize donor to donor variation in exosome batch production. In addition, the yield of CSSCs is also very poor, when compared to commercially available sources of BM-MSCs. Hence, the protocol to reprogram BM-MSCs to behave like CSSCs will provide sufficient cell yields for the production of therapeutic exosomes. Approximately, 0.5-1M stem cells per donor cornea can be expanded to 4-6M in 3 passages. Commercially available BMMSCs can be expanded from 1M to 80-120M in 3 passages. Hence, 20-30 folds higher cell yield is achieved by using BMMSCs versus CSSCs. However, CSSCs (cornea resident MSCs) have shown to be immensely effective in corneal wound healing that cannot be mimicked by the use of BMMSCs. Therefore, the priming of BMMSCs with CSSC-conditioned media reprograms BMMSCs into CSSC-like stem cells. This protocol will help produce 20-60 folds higher CSSC-like BMMSC cell yield and exosomes. While using CSSC-exosomes can help treat 8-10 corneas at a dose of 0.1-0.5 billion exosomes per eye, the priming protocol proposes to treat 20-60× i.e. 200-600 patients from a single donor cornea. Furthermore, by employing the 3D scalable cell culture process as described in the Examples 6 and 7 further amplification of the cell and exosome yield is achieved by an additional 5-10 folds. Hence, it can be inferred that the combination of CSSC-CM priming protocols with 3D expansion methods (as described in Examples 6 and 7) will yield 100-600 folds higher exosomes yield, allowing the treatment of approximately 1000-5000 patients per donor cornea.

The present disclosure discloses process of culturing MSC to obtain expanded MSC and a MSC-CM. Significant advantages include the scalability of the process as described herein along with the fact that the process is a xeno-free process, therefore, the process of the present disclosure gives a viable option of scalability for meeting the commercial requirements and also provides clinical grade end products in terms of MSC-CM. The MSC-CM is further processed to obtain clinical grade exosomes, secretome, and other cello-derived products which can be used for treating a condition selected from the group consisting of corneal disorders, liver fibrosis, and hyper-inflammatory conditions. As per the process disclosed in the present disclosure, high quality exosome yield of approximately 2 billion purified exosomes is obtained from approximately 1 million MSCs grown in 2D format (as per the Example 1 and 2). By culturing cells employing the process of the present disclosure 3D scalable platforms, at least 5-10 folds amplification can be obtained in exosome yield. As per the present disclosure, the exosome yield is scalable without impacting the production costs. Advantage in terms of total proteins, cell viability and quality can be observed in the Table 5 and Table 6.

I/We claim:

1. A process for obtaining an expanded primed mesenchymal stem cell population, said process comprising:
a) obtaining a population of mesenchymal stem cells;
b) culturing the population of mesenchymal stem cells in a culture medium comprising a corneal stromal stem cell derived-conditioned medium to obtain primed mesenchymal stem cells, wherein the corneal stromal stem cell derived-conditioned medium is obtained from culturing of corneal limbal stem cells; and
c) expanding the primed mesenchymal stem cells obtained in step (b) in a culture medium, to obtain an expanded primed mesenchymal stem cell population, and a mesenchymal stem cell derived-conditioned medium.

2. The process as claimed in claim 1, wherein the process is for obtaining a mesenchymal stem cell derived-conditioned medium.

3. The process as claimed in claim 1, wherein culturing the population of mesenchymal stem cells is done in a culture medium comprising a corneal stromal stem cell derived-conditioned medium in a volume percentage in a range of 5-50% with respect to the culture medium.

4. The process as claimed in claim 1, wherein expanding the primed mesenchymal stem cells is done in either a spheroid-based system or a microcarrier-based system, to obtain a population of expanded primed mesenchymal stem cells.

5. The process as claimed in claim 4, wherein expanding the primed mesenchymal stem cells is done in a spheroid-based system comprising steps of:
a) pelleting the primed mesenchymal stem cells obtained in step (b) of claim 1, to obtain a primed mesenchymal stem cell pellet;
b) resuspending the primed mesenchymal stem cell pellet in a suitable volume of a culture medium comprising MSC basal medium, to obtain a primed mesenchymal stem cell suspension;
c) processing the primed mesenchymal stem cell suspension to obtain primed mesenchymal stem cell spheroids having a density of mesenchymal stem cells in a range of 600-10,000 cells per spheroid; and
d) culturing the primed mesenchymal stem cell spheroids in a culture medium comprising MSC basal medium to obtain a population of expanded primed mesenchymal stem cells, and a mesenchymal stem cell derived-conditioned medium.

6. The process as claimed in claim 5, wherein the culture medium of step (b) and step (d) comprises methyl cellulose in a concentration range of 0.2-2% with respect to the culture medium.

7. The process as claimed in claim 5, wherein the culture medium of step (b) comprises methyl cellulose in a concentration range of 0.2-2% with respect to the culture medium.

8. The process as claimed in claim 5, wherein the culture medium of step (d) comprises methyl cellulose in a concentration range of 0.2-2% with respect to the culture medium.

9. The process as claimed in claim 4, wherein culturing is done in a microcarrier based system comprising steps of:
a) obtaining microcarriers comprising crosslinked alginate core and crosslinked gelatin surface;
b) suspending the microcarriers in a culture medium, to obtain a suspension;
c) seeding the suspension with the primed mesenchymal stem cells obtained in step (b) of claim 1; and
d) culturing the primed mesenchymal stem cells to obtain a population of expanded primed mesenchymal stem cells adhered to the microcarriers, and a mesenchymal stem cell derived-conditioned medium.

10. The process as claimed in claim 9, wherein the microcarriers are in a size ranging from 50-500 μm.

11. The process as claimed in claim 9, wherein the microcarriers comprise sodium alginate in the concentration range of 0.01-20% w/v, and gelatin in the concentration range of 0.1-20% w/v.

12. The process as claimed in claim 1, wherein the corneal stromal stem cell derived-conditioned medium is obtained by culturing of corneal limbal stem cells, said culturing comprises:
a) obtaining a limbal ring tissue from a human donor cornea;
b) mincing the tissue, to obtain fragments in the size ranging from 1 to 2 mm;
c) suspending the fragments in an incomplete medium, to obtain a suspension;
d) subjecting the fragments to digestion in the presence of at least one type of collagenase enzyme at a concentration range of 5-20 IU/μl with respect to the suspension, to obtain digested explants;
e) culturing the digested explants in a complete medium comprising 1-3% human platelet lysate for a period of 10-14 days, to obtain a population of corneal limbal stem cells; and
f) passaging the corneal limbal stem cells of step (e) for a period of 10-14 days, to obtain expanded corneal stromal stem cells, and a corneal stromal stem cell derived-conditioned medium.

13. The process as claimed in claim 1, wherein the population of mesenchymal stem cells is selected from the group consisting of human bone marrow-derived mesenchymal stem cells, adipose tissue-derived mesenchymal stem cells, umbilical cord-derived mesenchymal stem cells, Wharton jelly-derived mesenchymal stem cells, dental pulp derived mesenchymal stem cells, and induced pluripotent stem cells.

14. An expanded primed mesenchymal stem cell population obtained by the process as claimed in claim 1.

15. A mesenchymal stem cell derived-conditioned medium obtained by the process as claimed in claim 2.

16. A composition comprising the mesenchymal stem cell derived-conditioned medium as claimed in claim 15.

17. A composition comprising the expanded primed mesenchymal stem cell population as claimed in claim 14.

18. An exosome preparation obtained by a process comprising: (a) harvesting the mesenchymal stem cell derived-conditioned medium as claimed in claim 15, to obtain a secretome; (b) centrifuging the secretome, to obtain a pellet; (c) dissolving the pellet in a low serum xenofree media, to obtain a crude solution; (d) performing density gradient ultracentrifugation with the crude solution, to obtain a fraction comprising exosomes; and (e) purifying the fraction comprising the exosomes by size exclusion chromatography, to obtain an exosome preparation.

19. A composition comprising at least two components selected from the group consisting of: (a) the expanded primed mesenchymal stem cell population as claimed in claim 14, (b) the mesenchymal stem cell derived-conditioned medium as claimed in claim 15, and (c) the exosome preparation as claimed in claim 18.

20. A method for treating a condition selected from the group consisting of corneal disorders, liver fibrosis, and hyper-inflammatory conditions, said method comprising: (a) obtaining the exosomes as claimed in claim 18; and (b) administering the exosomes to a subject for treating the condition.

21. A method for treating a condition selected from the group consisting of corneal disorders, liver fibrosis, and hyper-inflammatory conditions, said method comprising: (a) obtaining the mesenchymal stem cell derived-conditioned medium as claimed in claim 15; and (b) administering a therapeutically effective amount of the conditioned medium to a subject for treating the condition.

22. A method for treating a condition selected from the group consisting of corneal disorders, liver fibrosis, and hyper-inflammatory conditions, said method comprising: (a) obtaining the expanded primed mesenchymal stem cell population as claimed in claim 14; and (b) administering a therapeutically effective amount of the expanded primed mesenchymal stem cell population to a subject for treating the condition.

23. A method for treating a condition selected from the group consisting of corneal disorders, liver fibrosis, and hyper-inflammatory conditions, said method comprising: (a) obtaining the composition as claimed in claim 19; and (b) administering a therapeutically effective amount of the composition to a subject for treating the condition.

24. The composition as claimed in any one of the claims 16, 17, or 19 for use in treating a condition selected from the group consisting of corneal disorders, liver fibrosis, and hyper-inflammatory conditions.

25. The expanded mesenchymal stem cell population as claimed in claim 14, or the mesenchymal stem cell derived-conditioned medium as claimed in claim 15, or the exosome preparation as claimed in claim 18, for use in treating a condition selected from the group consisting of corneal disorders, liver fibrosis, and hyper-inflammatory conditions.

26. The process as claimed in claim 9, wherein population of expanded primed mesenchymal stem cells adhered to the microcarriers is contacted with a dissolution buffer comprising sodium chloride and trisodium citrate to obtain a population of expanded primed mesenchymal stem cells.
